(19) United States
(12) Patent Application Publication
(10) Pub. No.: US 2006/0275270 A1
(43) Pub. Date: Dec. 7, 2006

(54) In vitro mucosal tissue equivalent

(76) Inventors: William L. Warren, Orlando, FL (US); Guzman Sanchez-Schmitz, Orlando, FL (US); Russell N. Higbee, Orlando, FL (US); Heather Fahlenkamp, Broken Arrow, OK (US); Donald Drake III, Orlando, FL (US); John G. Tew, Mechanicsville, VA (US)

Correspondence Address: MERCHANT & GOULD PC, P.O. BOX 2903, MINNEAPOLIS, MN, 55402-0903, US

(21) Appl. No.: 11/375,128

(22) Filed: Mar. 15, 2006

Related U.S. Application Data
(63) Continuation-in-part of application No. 11/116,234, filed on Apr. 28, 2005.
(60) Provisional application No. 60/565,846, filed on Apr. 28, 2004.
(60) Provisional application No. 60/752,034, filed on Dec. 21, 2005.

(30) Foreign Application Priority Data
Apr. 28, 2005 (WO) PCT/US05/14444

(51) Int. Cl. A61K 35/14; A61K 39/00; C12N 5/08; A61K 9/14

(52) U.S. Cl. 424/93.7; 424/184.1; 435/366; 435/372; 424/486

(57) ABSTRACT

The present invention relates to methods of constructing an integrated artificial immune system that comprises appropriate in vitro cellular and tissue constructs or their equivalents to mimic the normal tissues that interact with pathogens and vaccines in mammals. The artificial immune system can be used to test the efficacy of vaccine candidates in vitro and thus, is useful to accelerate vaccine development and testing drug and chemical interactions with the immune system.

### CROSS-REFERENCE TO RELATED APPLICATIONS

This application is a continuation-in-part of U.S. application Ser. No. 11/116,234, filed Apr. 28, 2005, which claims the benefit of priority of U.S. Provisional Application Ser. No. 60/565,846, filed Apr. 28, 2004. This application also claims the benefit of priority of International Application No. PCT/US2005/014444, filed Apr. 28, 2005. This application further claims the benefit of priority of U.S. Provisional Application Ser. No. 60/752,034, filed Dec. 28, 2005. Each of these applications is hereby incorporated by reference in their entirety.

### INTRODUCTION

1. Field of the Invention

The present invention is directed to a method for constructing an integrated artificial human tissue construct system and, in particular, construction of an integrated human immune system for in vitro testing of vaccines, adjuvants, immunotherapy candidates, cosmetics, drugs, biologics, and other chemicals. The artificial immune system of the present invention is useful for assessing the interaction of substances with the immune system, and thus can be used to accelerate and improve the accuracy and predictability of, for example, vaccine, drug, biologic, immunotherapy, cosmetic, and chemical development.

2. Background of the Technology

Despite the advent and promise of recent technologies, including combinatorial chemistry, high-throughput screening, genomics, and proteomics, the number of new drugs and vaccines reaching the market has not increased. In fact, the attrition rate within drug discovery programs is still over 90%.

The introduction of these new (and expensive) technologies has not reduced the lost opportunity costs associated with immunotherapy development; rather, these costs have increased. It is now estimated that almost $1.2 billion is required to bring a new drug to the market. This number, of course, includes all the cost of failures along the way from selecting a target to successful clinical research and an approved product The development and biological testing of human vaccines has traditionally relied on small animal models (e.g., mouse and rabbit models) and then non-human primate models. However, such small animal models are expensive and non-human primate models are both expensive and precious. Furthermore, there are many issues regarding the value of such animal studies in predicting outcomes in human studies.

A major problem remains the translation from test systems (animal or 2-dimensional (2D) cell culture) to human immunology. Successful transfer between traditional testing systems and human biology requires an intricate understanding of disease pathogenesis and immunological responses at all levels. Given worldwide health problems caused by known and emerging infec-tious agents and even potential biological warfare pathogens, it is time for a fresh approach to understanding disease pathogenesis, the development and rapid testing of vaccines, and insights gathered from such work.

The distributed immune system can be roughly divided into four distinct compartments: tissues and blood, mucosal tissues, body cavities, and skin. Because of ease of study, most is known about the tissue and blood compartment and its lymphoid tissues, the spleen and lymph nodes.

However, the largest compartment is the MALT (mucosa-associated lymphoid tissue). Mucosal surfaces serve a wide range of functions, including exchange of gases (lungs), nutrient transport (digestive tract), sensory surfaces (nose, mouth, throat), and reproductive signals. Mucosal immunity is important for several reasons. First, the vast majority of human pathogens, including many of the leading infectious disease killers, initiate infections at mucosal surfaces, the largest routes of entry into the body. Additionally, stimulation of a mucosal immune response can result in production of protective B and T cells in both mucosal and systemic environments, so that infections are stopped or significantly hindered before they enter the rest of body. Significantly, bioterrorism relies on entry of agents through mucosal surfaces, where pathogens or toxins are primarily encountered, not as injections.

Because of its large surface area and exposure to the outside world, the mucosal system is more vulnerable to infection than other body components (Newberry & Lorenz (2005) Immunol Rev 206, 6-21). As an example, the digestive tract has roughly 10¹⁴ commensal organisms and frequently encounters pathogens. Furthermore, an additional challenge for the gut-associated lymphoid system is that typical food antigens should be tolerated while pathogenic antigens should induce vigorous immune responses. A hallmark of the mucosal immune system is the production of secretory immunoglobulin A (IgA). MALT plasma cells secrete primarily dimeric IgA in an IgA₁:IgA₂ ratio of 3:2; whereas, IgA secreted in the tissue and blood compartment is primarily monomeric IgA in an IgA₁:IgA₂ ratio of 4:1. IgA₂ is more resistant to proteolysis by pathogens than IgA₁ (http://microvet.arizona.edu/Courses/MIC419/Tutorials/bigpicture.html).

The mammalian immune system uses two general adaptive mechanisms to protect the body against environmental pathogens. When a pathogen-derived molecule is encountered, the immune response becomes activated to ensure protection against that pathogenic organism.

The first immune system mechanism is the non-specific (or innate) inflammatory response. The innate immune system appears to recognize specific molecules that are present on pathogens but not on the body itself.

The second immune system mechanism is the specific or acquired (or adaptive) immune response. Innate responses are fundamentally the same for each injury or infection; in contrast, acquired responses are custom-tailored to the pathogen in question. The acquired immune system evolves a specific immunoglobulin (antibody) response to many different molecules present in the pathogen, called antigens. In addition, a large repertoire of T cell receptors (TCR) is sampled for their ability to bind processed forms of the antigens bound to major histocompatibility complex (MHC, also known as human leukocyte antigen, HLA) class I and II proteins on the surface of antigen-presenting cells (APCs), such as dendritic cells (DCs).

The immune system recognizes and responds to structural differences between self and non-self proteins. Proteins that the immune system recognizes as non-self are referred to as antigens. Pathogens typically express large numbers of complex antigens.

Acquired immunity is mediated by specialized immune cells called B and T lymphocytes (or simply B and T cells). Acquired immunity has specific memory for antigenic structures; repeated exposure to the same antigen increases the response, which increases the level of induced protection against that particular pathogen.

B cells produce and mediate their functions through the actions of antibodies. B cell-dependent immune responses are referred to as “humoral immunity,” because antibodies are found in body fluids.

T cell-dependent immune responses are referred to as “cell mediated immunity,” because effector activities are mediated directly by the local actions of effector T cells. The local actions of effector T cells are amplified through synergistic interactions between T cells and secondary effector cells, such as activated macrophages. The result is that the pathogen is killed and prevented from causing diseases.

The functional element of a mammalian lymph node is the follicle, which develops a germinal center (GC) when stimulated by an antigen. The GC is an active area in a lymph node, where important interactions occur in the development of an effective humoral immune response. Upon antigen stimulation, follicles are replicated and an active human lymph node may have dozens of active follicles, with functioning GCs. Interactions between B cells, T cells, and FDCs take place in GCs. Various studies of GCs in vivo indicate that the following events occur there, including immunoglobulin (Ig) class switching, rapid B cell proliferation (GC dark zone), production of B memory cells, accumulation of select populations of antigen specific T cells and B cells, hypermutation, selection of somatically mutated B cells with high affinity receptors, apoptosis of low affinity B cells, affinity maturation, induction of secondary antibody responses, and regulation of serum immunoglobulin G (IgG) with high affinity antibodies. Similarly, data from in vitro GC models indicate that FDCs are involved in stimulating B cell proliferation with mitogens and it can also be demonstrated with antigen (Ag), promoting production of antibodies including recall antibody responses, producing chemokines that attract B cells and certain populations of T cells, and blocking apoptosis of B cells.

Similar to pathogens, vaccines function by initiating an innate immune response at the vaccination site and activating antigen-specific T and B cells that can give rise to long term memory cells in secondary lymphoid tissues. The precise interactions of the vaccine with cells at the vaccination site and with T and B cells of the lymphoid tissues are important to the ultimate success of the vaccine.

Almost all vaccines to infectious organisms were and continue to be developed through the classical approach of generating an attenuated or inactivated pathogen as the vaccine itself. This approach, however, fails to take advantage of the recent explosion in our mechanistic understanding of immunity. Rather, it remains an empirical approach that consists of making variants of the pathogen and testing them for efficacy in non-human animal models.

Advances in the design, creation and testing of more sophisticated vaccines have been stalled for several reasons. First, only a small number of vaccines can be tested in humans, because, understandably, there is little societal tolerance for harmful side effects in healthy people, especially children, exposed to experimental vaccines. With the exception of cancer vaccine trials, this greatly limits the innovation that can be allowed in the real world of human clinical trials. Second, it remains challenging to predict which epitopes are optimal for induction of immunodominant CD4 and CD8 T cell responses and neutralizing B cell responses. Third, small animal testing, followed by primate trials, has been the mainstay of vaccine development; such approaches are limited by intrinsic differences between human and non-human species, and ethical and cost considerations that restrict the use of non-human primates. Consequently, there has been a slow translation of basic knowledge to the clinic, but equally important, a slow advance in the understanding of human immunity in vivo.

The artificial immune system (AIS) of the present invention can be used to address this inability to test many novel vaccines in human trials by instead using human tissues and cells in vitro. The AIS enables rapid vaccine assessment in an in vitro model of human immunity. The AIS provides an additional model for testing vaccines in addition to the currently used animal models.

Attempts have been made in modulating the immune system. See, for example, U.S. Pat. No. 6,835,550 B1, U.S. Pat. No. 5,008,116, Suematsu et al., (Nat Biotechnol, 22, 1539-1545, (2004)) and U.S. Patent Application No. 2003/0109042.

Nevertheless, none of these publications describe or suggest an artificial (ex-vivo) human cell-based immune-responsive respiratory mucosal model system. The present invention comprises such a system and its use in assessing the interaction of substances with the immune system.

### DESCRIPTION OF THE INVENTION

The present invention comprises an ex-vivo human cell-based immune-responsive respiratory mucosal model system that can supplement animal models in the study of immunotherapy efficacy and safety. The mucosal tissue equivalent (MTE) of the present invention will help our understanding of infectious disease pathogenesis, speed up development and testing of vaccines and drugs, and allow the redesign/optimization of vaccine or drug formulations before animal testing or clinical trials. The present invention comprises a minimal tissue engineered immune system that mimics the functions of the respiratory mucosal immune system.

The present invention concerns the development of accurate, predictive in vitro models to accelerate vaccine testing, allow collection of more informative data that will aid in redesigning and optimizing vaccine formulations before animal or clinical trials, and raise the probability that a vaccine candidate will be successful in human trials. The present invention comprises a new in vitro mucosal tissue equivalent (MTE) that can be used as a diagnostic tool to decrease the cycle time yet enhance mechanistic insights resulting from rounds of vaccine testing and reformulation. The end result is clinically relevant information earlier in the vaccine development process, thereby saving potentially hundreds of millions of dollars in misdirected R&D and lost opportunity costs.

A given immune response against a pathogen or vaccine reflects, in large part, the quality of the primary interaction with specific cells at the site of initial exposure, the innate immune response, and the resulting effector cells that activate a subsequent adaptive immune response. The present invention comprises a modular, integrative immune-functional in vitro MTE system. The system comprises two components: (1) a mucosal exposure site (MES) lacking mucosa-associated lymphoid tissue equivalent (MALTE), which is also suitable for exploring innate immune responses; and (2) a MES containing MALTE, which is suitable for exploring more complex immune responses such as antigen presentation in situ and antibody production. In other embodiments, the MTE may comprise differing epithelial cell sources, depending on specific needs.

In embodiments of the present invention, the 3D endothelial cell construct is modified to include a basic architecture comprising a well-based 3D membrane scaffolding format, with a confluent vascular endothelium on one side, a respiratory mucosal epithelium on the other side, and matrix-embedded fibroblasts in between. In this embodiment of the mucosal tissue equivalent system (MTE), a heterogeneous tissue construct is prepared, comprising fibroblasts embedded within the matrix; a layer selected from the group consisting of an epithelial layer (such as nasal epithelium, oral epithelium, respiratory epithelium, gastrointestinal epithelium, conjuctival epithelium, and urogenital epithelium), an epithelium, a mucosal epithelium, and a confluent respiratory mucosal epithelium, attached to one side of the matrix; and a layer selected from the group consisting of an endothelial layer, an endothelium, a vascular endothelium, and a confluent vascular endothelium, attached to the other side of the matrix. The fibroblast-embedded matrix may further comprise cells selected from the group consisting of T cells, B cells, macrophages, monocytes, mast cells, dendritic cells, and follicular dendritic cells. The mucosal tissue equivalent system may further comprise a lymphoid follicle or a germinal center. The mucosal tissue equivalent system may be organized in a well or a multi-well format.

The matrix used in the mucosal tissue equivalent system may be selected from the group consisting of a collagen membrane, hydrogels, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM (such as small intestine submucosa and urinary bladder mucosa).

Methods of preparing MTEs of the present invention are also provided.

We have previously established a 3D endothelial cell construct for monocyte migration and DC/macrophage differentiation that has been used to test various antigens. The construct allows the in vitro development in 3D of a MES module with a supportive biocompatible micro-environment for a variety of cells to proliferate, differentiate, and migrate in a manner that recapitulates normal mucosal immunophysiology.

The MTE model allows the study of complex localized mucosal adaptive responses, in addition to the more common responses studied from tissue-migrated antigen-primed DCs arriving at draining lymph nodes. The MTE will also enable the study of an innate immune response against respiratory pathogens and vaccines. This in vitro system is supplied with sufficient nutrients and gases to enable survival in culture for a period of several weeks, a time consistent with development of antigen-specific adaptive immune responses in vivo. In another embodiment, to ensure specific antibody production, free, solubilized antigen can be provided to ensure the occurrence of T-independent B cell activation.

In an embodiment of the present invention, the MTE can be prepared in a well-based format to facilitate high-throughput and mechanization.

In embodiments of the present invention, the MTE system is prepared using the MES module as the template and increasing the cellular complexity by including pre-selected T cells, B cells, and FDCs inside the matrix (Tew et al. (1997) Immunol Rev 156, 39-52). The purpose is to establish inside the MES a self-organizing, functionally equivalent GC, mimicking the mucosa-associated lymphoid tissue functionality and complex structure observed in vivo in bronchus-associated lymphoid tissue (BALT).

As an example, an approach to generating a lymphoid follicle inside the MES module (similar to that used for the introduction of fibroblasts) is a step-by-step assembly process, starting with the 3-dimensional (3D) scaffolding construction in a membrane format by means of fast matrix congealing of a viable cell mix of pre-selected T and B cells, FDCs, and fibroblasts, followed by a more rapidly confluent endothelial and epithelial monolayers on the top and bottom of the membrane structure. Transendothelial migration of monocytes inside the construct completes the MTE system.

In another embodiment, lateral microinjection of the lymphoid follicle cell mix inside the MES can be used. The general idea is to maintain the cellular composition and complexity of the MTE, without compromising the possible need for defined tissue niches, as seen in vivo (Brandtzaeg & Johansen (2005) Immunol Rev 206, 32-63). Thus, for a complete respiratory MTE, professional antigen presentation can be performed by monocyte-derived dendritic cells (DCs), macrophages, or resident B cells, similar to that observed in vivo.

While T cells are necessary for B cell responses to T cell-dependent antigens, they are not sufficient for the development of fully functional and mature antibody responses that are required with most vaccines. FDCs provide important assistance needed for the B cells to achieve their full potential.

Humoral responses in vaccine assessment can be examined using the artificial immune system of the present invention. Accessory functions of follicular dendritic cells and regulation of these functions are important to an understanding of fully functional and mature antibody responses. Important molecules have been characterized by blocking ligands and receptors on FDCs or B cells. FDCs trap antigen-antibody complexes and provide intact antigen for interaction with B cell receptors (BCRs) on GC B cells; this antigen-BCR interaction provides a positive signal for B cell activation and differentiation. Engagement of CD21 in the B cell co-receptor complex by complement derived FDC-CD21L delivers an important co-signal. Coligation of BCR and CD21 facilitates association of the two receptors and the cytoplasmic tail of CD19 is phosphorylated by a tyrosine kinase associated with the B cell receptor complex. This co-signal dramatically augments stimulation delivered by engagement of BCR by antigen and blockade of FDC-CD21L reduces the immune responses 10- to 1,000-fold.

The interactions between B cells, T cells, and FDCs that occur in GCs result in stimulation of antibody specific B cells, immunoglobulin class switching, somatic hypermutation, and the selection of high affinity B cells responsible for affinity maturation and the production of high quality antibody.

In embodiments of the present invention, GCs are incorporated in the design of the MTE to facilitate examination of humoral responses to vaccines. The GCs contain large proliferating B lymphocytes interspersed with macrophages, DCs and FDCs. The GC is a site of intense B cell activation and differentiation into plasma cells and memory cells. In an embodiment of the present invention, GCs are incorporated in the design of an artificial immune system (AIS) to examine immune (especially humoral) responses to vaccines and other compounds.

In an embodiment of the present invention, development of an in vitro GC adds functionality to an AIS, in that it enables generation of an in vitro human humoral response by human B cells that is accurate and reproducible without using human subjects. The invention also permits evaluation of vaccines, allergens, and immunogens and activation of human B cells specific for a given antigen, which can then be used to generate antibodies. In an embodiment of the present invention the function of the in vitro GC is enhanced by placing FDCs and other immune cells in a 3D ETC; FDCs appear more effective over a longer time (antibody production is sustained for up to 14 days).

The present invention comprises placing FDCs in an engineered tissue construct, such as a collagen cushion, microcarriers, inverted colloid crystal matrices, or other synthetic or natural extracellular matrix material, where they can develop in 3D. FDCs in the in vivo environment are attached to collagen fibers and do not circulate, as most immune system cells do. Thus, placing FDCs in, for example, a collagen matrix is more in vivo-like. In other embodiments, in addition to creating the GC in 3D, a follicle with GC, T cell zones, and B cell zones in the scaffolding provided by the ETC matrix is developed. Immobile FDCs form a center and the chemokines they secrete may help define the basic features of an active follicle.

Being able to reconstruct follicles where important events for productive humoral immune responses take place is of importance in assessing vaccines. For example, it is not uncommon to find non-responders to particular vaccine; such people may be put at risk when given a live vaccine. In an embodiment of the present invention, such non-responders can be identified by establishing a model of their immune system in vitro (i.e., using their cells) and determining their non-responsive or poorly responsive state before they were challenged with a live vaccine capable of causing harm. In another embodiment of the present invention, immunomodulators that could convert such poor responders into good responders can be identified and formulated for use in vivo. Such an approach has the potential to reduce vaccine development times and costs and to improve vaccine efficacy and reduce reliance on animal models. In addition, some therapeutic agents and industrial chemicals are toxic to the immune system and in other embodiments an in vitro immune system comprising in vitro germinal centers could be used to assess immunotoxicity and the effects of allergens in the context of a model human immune system. The present invention can also be used to assess therapeutic agents that could convert immune responders to non-responders, which would be invaluable for the treatment of antibody-mediated autoimmune disorders.

The present invention comprises immunological constructs to mimic normal immunophysiology. The artificial immune system of the present invention comprises the incorporation of a 3D microstructure, immune cells, a vascular endothelium, a respiratory epithelium, and a lymphoid follicle. The system also enables in situ cytokine analysis, within the constructs.

Each of the constructs has a 3-dimensional (3D) architecture that supports and maintains tissue function. Such a 3D tissue construct permits heterologous cell-cell interactions and impacts the differentiation of DC precursors, including monocytes, in a manner that more closely mimics an intact human system than is observed in 2D culture.

An important component of the construction of the MTE, mirroring a step in the induction of immunity during vaccination, is the delivery of antigen to APCs. APCs, especially DCs, engulf and process the antigen and then traffic to the MALTE or local lymph node where they present their antigen to T and/or B lymphocytes to initiate immune responses.

DCs are diverse in nature; they reside in host tissues and many populations of DCs found in the blood as precursors can be rapidly recruited across the endothelial lining of blood vessels. The endothelium provides signals to the DCs while passing through a tissue. Which DC precursors enter a tissue, and therefore which types of DCs may respond to a vaccine formulation or pathogen, is controlled partly by the endothelium. Endothelial cells can modulate their expression of adhesion molecules and chemokines, for example, to regulate entrance of DCs and other cell types, including classical inflammatory cells such as neutrophils (Smits et al. (1996) J Dairy Sci 79, 1353). An important component of any vaccine exposure site model is the inclusion of vascular endothelial cells that orchestrate which precursor cell populations are recruited to the site of vaccination.

The incorporation of a respiratory epithelium is an important consideration because, unlike a skin epithelium, the respiratory epithelium forms an intercommunicating network with APCs sampling the respiratory mucocilliary blanket and luminal milieu, in which signals are routinely exchanged in dynamic interactions. Respiratory epithelial cells produce a range of immune regulating cytokines and actively take part in the immune response.

The germinal center (GC) is a “hot spot” where important interactions take place in developing an effective humoral immune response. Interactions between B cells, T cells, and FDCs take place in GCs. These interactions result in stimulation of antibody-specific B cells, immunoglobulin class switching, somatic hypermutation, and the selection of high-affinity B cells responsible for affinity maturation and the production of high quality antibodies. The FDCs provide assistance to the B cells so that they achieve their full potential. Such accessory functions of FDCs and regulation of these functions are important to an understanding of fully functional and mature antibody responses that occur in the associated lymphoid tissues in the body. Immobile FDCs producing chemokines help define the basic architectural features of an active follicle. In embodiments of the present invention, the constructs use natural self-assembly processes in which the cells provide the natural cues as much as possible. The MTE can also incorporate the production of secretory immunoglobulin A, which attaches to the mucus overlying the respiratory epithelium, where it can neutralize pathogens or their toxins. The immunological constructs comprising the MTE include the MES and the mucosa-associated lymphoid tissue equivalent (MTE); they are modular in nature. Each module can function independently as a minimal model of localized mucosal immune response against antigens, vaccines, pathogens, and inflammatory signals.

In further embodiments of the present invention, by changing the epithelial cell types, the MTE can be customized for assessing vaccines at all sites of pathogen entry, nasal, oral, respiratory, gastrointestinal, conjunctival, and urogenital.

Embodiments of the present invention using well-based format permit high-throughput analysis of, e.g., various antigen/adjuvant combinations when assessing vaccine formulations.

An important component of the immune response, mirroring an important step in the induction of immunity, is the capture of antigens by APCs. APCs engulf and process antigen and then may traffic to the closest GC in the MTE, where they interact with T and/or B cells to initiate antigen-specific immune responses or traffic to the LTE.

An important aspect of the sub-epithelial region is the reproduction of this process by allowing autonomous generation of resident macrophages and APCs, such as migratory DCs, with as little artificial (mechanical or exogenous cytokine) intervention as possible.

The construct has a 3D architecture capable of supporting and maintaining normal tissue function. For the MTE to act as a respiratory mucosal site with a capacity to provide adaptive immune responses, it is autonomous in the sense that antigen-presenting cells (APCs) are generated in vitro antigen from migratory monocytes. It is known that blood monocytes can extravasate from the vasculature, colonizing tissues and becoming resident macrophages and migratory dendritic cells (DCs), depending on endogenous signals (Randolph et al. (1998) Science 282, 480-483; Randolph et al. (2002) J Exp Med 196, 517-527; Randolph et al. (1999) Immunity 11, 753-761).

From previously developed 3D tissue constructs, placing or flowing monocytes along confluent vascular endothelia, allows colonization in in vitro models, providing autonomous capacity to generate APCs. This process mimics in vivo human physiology (Randolph et al. (1998) Proc Natl Acad Sci USA 95, 6924-6929); when these APCs are tested, we can achieve better immune responses to known antigens than those observed in commonly used 2D cultures.

In embodiments of the present invention, the mucosa-associated lymphoid tissue feature of the MTE also comprises T and B cells and follicular dendritic cells (FDCs) within the 3D construct. While T cells are necessary for B cell responses to T cell-dependent antigens, they are not sufficient for the mature antibody responses normally associated with vaccines. For that, FDCs can be used to provide the assistance needed for B cells to achieve their full potential, as shown in functional germinal centers (GCs) developed in vitro (Okazaki et al. (2003) Plast Reconstr Surg 112, 784-792). Providing the B and T cells in the presence of immobile FDCs in the matrix allows organization into secondary lymphoid follicles (LFs). After antigen challenge and immune complex formation, a network with mobile B and T cells occurs, as is seen in experiments using a murine system. The GC region of the lymphoid follicle is where important B and T cell interactions occur with FDCs leading to effective humoral immune responses, including immunoglobulin production, class switching, somatic hypermutation, and affinity maturation. By forming the secondary follicles, this establishes a functional element of the human respiratory MTE. Under antigenic stimulation, antibody-producing GCs develop.

The MTE of the present invention enables the study of the development of mucosal protective vaccines. The respiratory system in particular, with its great surface area and large population of immune cells provides an attractive target for immunization. Novel vaccines to protect inaccessible human mucosal surfaces and secretions (such as the genital tract or breast milk) may be delivered to the lung, gut or nasal tract and protection may be disseminated throughout the mucosa-associated lymphoid tissue (MALT).

The MTE of the present invention also enables one to determine whether a patient is a poor or non-responder to a vaccine. In this embodiment of the invention, vaccines are administered to the epithelial or mucosal epithelial cells of the MTE (prepared from the patient's own cells) and the immune response to the vaccine is analyzed.

In another embodiment of the invention, methods for identifying agents that can convert a patient that is a poor or non-responder to a vaccine to a good responder to a vaccine are provided. In this embodiment, prior to administering the vaccine to the epithelial cells or mucosal epithelial cells, an immunomodulator is administered. Then, the patient's response to the vaccine is analyzed to determine whether the patient has been converted to a good responder to the vaccine.

The MTE of the present invention also enables the study of immunogenicity of agents. Methods of testing for the immunogenicity of an agent comprise applying an antigen to the epithelial cells in the epithelial layer or mucosal epithelial cells in the mucosal epithelium of the MTE and analyzing the immune response. The agent can be selected from vaccines, respiratory pathogens, allergens, drugs and immunogens.

The MTE of the present invention is also useful for identifying agents useful for treating an antibody-mediated autoimmune disorder in a patient. In this regard, and MTE is prepared using the patients own cells, and an agent is administered to the epithelial or mucosal epithelial cells. The amount of autoimmune antibodies present in the MTE is subsequently quantified. If the amount of autoimmune antibodies present in the MTE tested with the agent is reduced, as compared to an MTE not challenged with the agent, then the agent may be useful for treating an antibody-mediated autoimmune disorder in that patient.

The immune response to pathogens and the efficacy of vaccines depends, in large part, on the quality of the initial interactions with cells at the site of infection or vaccination. To create a useful model of mucosal disease pathogenesis and vaccination, it is important to construct an artificial vaccination site in combination with an associated lymphoid tissue in vitro.

The artificial tissue of the present invention acts as a functional in vitro mucosal immune system. It comprises a modular and integrative system consisting of a MES, a mucosa-associated lymphoid tissue equivalent (MALTE), resembling the bronchus-associated lymphoid tissue (BALT), and a lymphoid tissue equivalent (LTE).

The key steps of an immune reaction comprise immune cell trafficking, antigen processing and presentation, and lymphocyte activation. In the artificial immune system of the present invention, lymphocyte activation occurs in an artificial lymph node, referred to as the lymphoid tissue equivalent (LTE) or artificial lymphoid tissue or a mucosal-associated lymphoid tissue (MALTE). An MES with MALTE is essentially a functional MTE. This in vitro system is supplied with sufficient nutrients and gases to enable survival of the cells/tissues in culture for several weeks.

In the artificial immune system of the present invention, the MES immunological construct replicates immune cell trafficking and antigen processing. It comprises a confluent vascular endothelium on one side and a respiratory epithelium on the other, separated by a, for example, collagen membrane. In embodiments of the present invention, a diverse assortment of primary cells, such as blood-derived hematopoietic cells and fibroblasts, can be included to mimic the cellular composition and complexity of the respiratory immune environment in vivo. Peripheral blood mononuclear cells (PBMC) can be placed or flowed along the vascular endothelium, where monocytes naturally extravasate and differentiate into APCs such as DCs, or reside in the tissue as alveolar macrophages (Clara cells). If APCs of the correct subtype and maturation state are present, they accept a challenge pathogen or vaccine candidate for testing.

Recently, it has been shown that DCs can extend their dendrite processes through epithelial tight junctions into the lumen and can sample its content (e.g., pathogens, such as bacteria). The DCs can also take any such pathogens below the epithelial surface without altering epithelial tight-junction permeability. As subepithelial DCs are widely distributed below the mucosal epithelial surface, this mechanism of immunosurveillance is thought to play an important role in mucosal immune responses. The present invention enables the study of such DC sampling and antigen processing routes. MES-derived APCs can be integrated with the MTE or LTE to assess their immunologic capabilities. In another embodiment, solubilized antigen can be introduced into the MTE for direct B cell processing.

In a typical mucosal immune response, antigens (from, e.g., microorganisms) are captured by DCs; these DCs migrate to adjacent lymphoid follicles. In the artificial immune system of the present invention, another cell type that can be integrated into the MTE is the FDC, which can to help form GCs. Interactions between B cells, T cells, and FDCs take place in GCs.

In this regard, additional embodiments of the invention comprise methods of developing in vitro lymphoid follicles or germinal centers. This method comprises embedding follicular dendritic cells in synthetic or natural extracellular matrix (ECM) material in condition in which they can develop a three-dimensional germinal center. In an alternative embodiment, methods of developing in vitro lymphoid follicles are provided, comprising embedding follicular dendritic cells in synthetic or natural extracellular matrix (ECM) material in condition in which they can develop a three-dimensional lymphoid follicles. In these methods, the synthetic or natural extracellular matrix may be a collagen cushion, microcarriers, inverted colloid crystal matrices, collagen membranes, hydrogels, poly(methyl methacrylate, poly(lactide-co-glycolide), polytetrafluoroethylene, poly(ethylene glycol dimethacrylate) hydrogels (PEGDAQ or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM (such as small intestine submucosa and urinary bladder mucosa).

An embodiment of the invention comprises confluent and viable endothelial cells on a collagen membrane structure. In other embodiments, membrane-based models can be incorporated into a well-based format that also allows incorporation of ancillary cells such as fibroblasts, FDCs, T and B cells.

In embodiments of the present invention, T and B cells, DCs and FDCs are loaded in collagen matrices having 3D infrastructure for cell residence, migration, and interaction. An advantage of such a model is the potential for co-migration of T cells, B cells, and FDCs in a porous environment. Enhanced migration makes T-B cell interaction more rapid.

In an embodiment of the present invention, a 3D heterogeneous MES model comprises cells on the top (epithelium) and bottom (endothelium), as well as within the matrix (fibroblasts) of the tissue construct. This embodiment provides an improvement over previously established 3D endothelial-only constructs used in studies of transendothelial migration of monocytes with differentiation to DCs and macrophages. The 3D MES model can be used to observe normal mucosal APC immunophysiology against various antigens.

In embodiments of the present invention, to improve the viability of the 3D tissue constructs, dialysis membranes are incorporated into the design of the AIS to reduce the need for media exchanges. By using dialysis membranes in the LTE, the incubation well can be designed to allow small molecules to pass freely across the membrane where as larger molecules, such as proteins, antibodies, and cytokines are retained.

Vaccine recipients typically have both naïve B cells and naïve T cells when given primary immunizations. To model such primary responses in vitro in an artificial immune system of the present invention, the artificial MTE should contain both naïve B cells and naïve T cells.

In an embodiment of the present invention, high throughput testing samples in an integrated MTE with an optional LTE can be effected using a multi-well-based format described here. The system comprises two components, the MTE and LTE. Each component of the system is treated separately and combined in the final step of testing if desired.

### BRIEF DESCRIPTION OF THE FIGURES

FIG. 1(A) is a schematic representation of an LTE in which T and B cells are cultivated together on microcarriers and then transferred to a porous container

FIG. 1(B) is a schematic representation of an LTE in which T and B cell are cultivated on separate microcarriers and then brought together in a porous container.

FIG. 1(C) is a schematic representation of an LTE in which separate T and B cell microcarriers are cultivated on separate microcarriers and then brought together in a porous container with separate compartments.

FIGS. 2(A) and 2(B). Practical considerations in AIS design.

FIG. 3 shows HUVEC cells growing on protasan/collagen matrix on a nylon mesh. High-magnification SEM of the nylon membrane and interspersed Protasan/collagen matrix material is shown in the top image. Seeding of the primary layer of HUVEC cells was accomplished on an inverted membrane (left, Side 1), then 24 hours later, brought to an upright position (right, Side 2) where the second layer was applied. Phase contrast images of each plane of HUVEC cells is shown in the center two lower images, with the left being the first layer, and the right being the second layer applied.

FIG. 4 shows mockup of digitally printed lymph node (left panel) and a retinal image of vasculature (right panel).

FIG. 5 shows image of microbeads fabricated from lymphoid ECM (80% w/w) and Protasan (20% w/w) by flash freezing, freeze drying, and gelation with tripolyphosphate.

FIG. 6 shows an additional embodiment involving ‘templating’ the LTE using native human stromal cells in a manner similar to that reported by researchers attempting to create an in vitro artificial thymus (Poznansky, et al., Nat. Biotechnol. 18:729-734, (2000)).

FIG. 7 shows a schematic of a bioreactor.

FIG. 8 is a plan view of an example integrated bioreactor that shows micromachined endothelial pathways with high contact area (left panel) beneath the VS and LTE ETCs (right panel).

FIG. 9 shows a laminate based insert whereas a larger milled tubular design is incorporated in to the design illustrated in FIG. 14.

FIG. 10 shows an example microfluidic bioreactor with optical diagnostics on microfluidic backplane.

FIG. 11 shows cross sectional views of direct deposition in the AIS device.

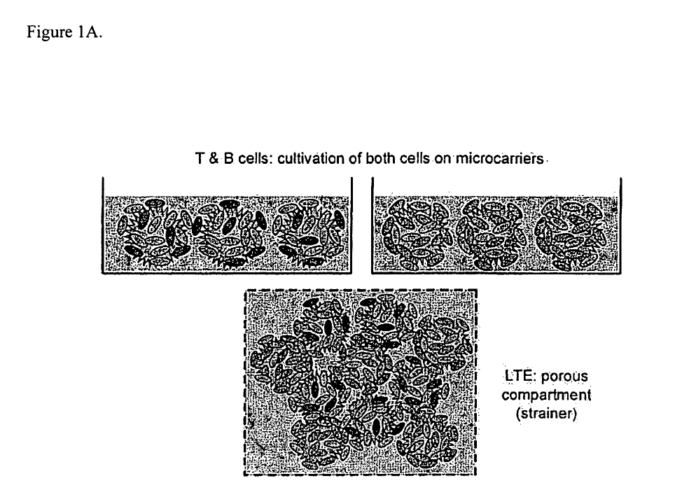

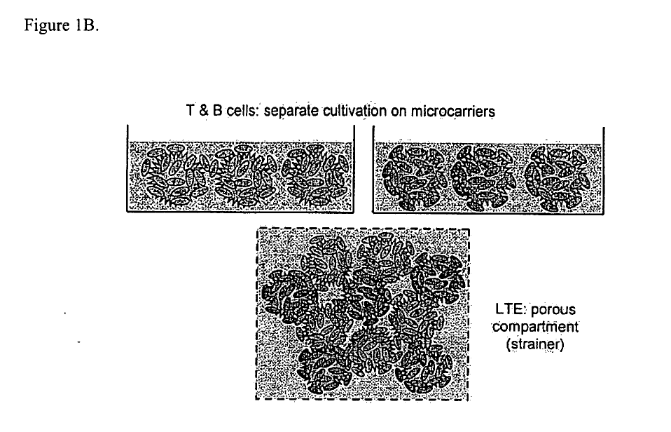

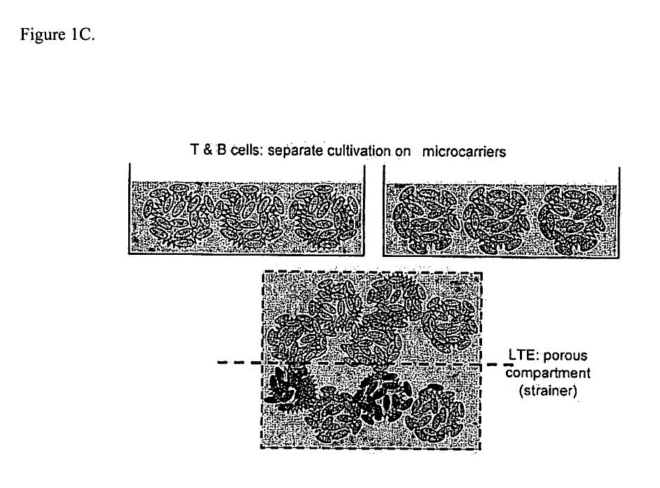

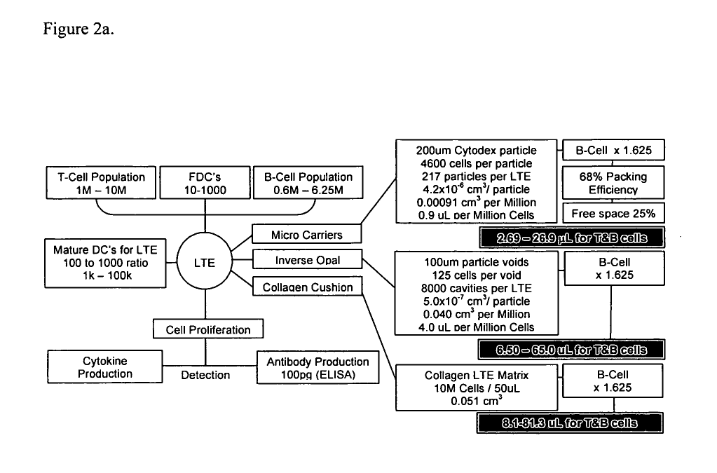

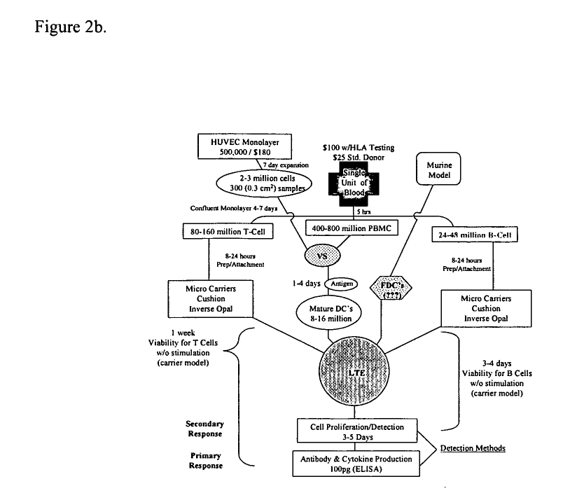

Various biomaterial structures can be incorporated as constituents of the artificial immune system (e.g., bio concrete, colloidal particles, ECM gels, collagen gels, microcarriers). For example, a polymeric mesh rebar can be deposited layer by layer directly in the recessions of the VS and LTE areas. In such a design, it is preferred to have the lower plate of the AIS unit made of polyacrylate, polystyrene, or another transparent plastic sensitive to DM, to allow the mesh rebar to attach to the plate. In this embodiment, the surface is micro-patterned using KOH in a manner similar to the ESC scaffolds. Fibrin gel matrix bearing all necessary nutrients and cytokines can be used to coat the threads of the mesh as a thin film, leaving sufficient space for cell accommodation and motion.

FIG. 12 shows an example microfluidic bioreactor in separate layers.

FIG. 13 shows an assembled microfluidic bioreactor.

FIG. 14 is a schematic diagram of perfused bioreactor system with the associated external pumps for vascular loops and external media reservoirs. The AIS bioreactor can be operated in semi-batch or continuous mode.

FIG. 15 shows membranes between thin metal (e.g., stainless steel) rings. Using such a crimping method, biological membranes can be supported without use of adhesives and can be pressed into a disk with thickness profile of about 400 μm or less.

FIG. 16 is a schematic showing the fabrication of a 3-layer planar waveguide.

FIG. 17 shows an example device comprising a perfusion bioreactor, an ELISA chip with integrated optical waveguides, microfluidic backplane to connect and allow swapping of devices and microfluidic connectors for external pumps and reservoirs.

FIG. 18 is a picture of synthetic and natural membranes supported by stainless steel rings.

FIG. 19 shows images of an ultra-short pulse laser micromachined planar optical waveguides integrated into microfluidic channel. Left panel: Tapered port for fiber optic coupling. Middle panel: microfluidic channel intersection of planar waveguide (source off). Right panel: microfluidic channel intersection of planar waveguide (source on, entering from right).

FIG. 20 shows an embodiment of the MaAIS.

FIG. 21 shows laser machined integrated optical waveguides: n1 represents the refractive index of the waveguide core, n2 is the cladding index.

FIG. 22 shows an example bioreactor construction with collagen membranes on rings and support matrix. Panel A shows a bioreactor design. Panel B shows progression from the whole bioreactor to the level of the collagen matrix cushion within the mesh. Panel C shows the assembly of the bioreactor under sterile conditions, after the HUVEC cells have reached confluence on the collagen cushion. Once assembled, media flow can be initiated.

FIG. 23 shows an example microfluidic bioreactor with optical diagnostics on microfluidic backplane.

FIGS. 24(A) and 24(B) illustrate well-based embodiments of the present invention, suitable for automation.

FIG. 25 illustrates a method of mounting an ECM membrane using concentric rings that can be used in a well-based format.

FIG. 26 illustrates a bioreactor.

FIGS. 27(A) and 27(B) illustrate integration of scaffolds in a 96-well format.

FIG. 28 shows how the VS and LTE constructs can be integrated into a well-based format in which the VS is used in a filter plate and the LTE is placed into the acceptor wells. The VS fits over the LTE in the design illustrated.

FIG. 29. High throughput testing using the integrated AIS can be accomplished using a static 96-well format, illustrated in this figure. The AIS of this embodiment comprises two parts, the VS and LTE. Each part is prepared separately and combined in the final step of testing. The simplicity of the system facilitates automation. Furthermore, the 96-well format, or other well-based formats, typically used in laboratory automation can accommodate these embodiments of the AIS.

FIG. 30. A representation of a VS model that can be used as a skin equivalent and how it can be tested with an allergen.

FIG. 31. Introduction of ancillary cells into a 3D construct.

FIG. 32. Schematic representation of a mucosal exposure site (MES)

FIG. 33. Schematic representation of the mucosal tissue equivalent (MTE).

FIG. 34. Simple tissue constructs based on endothelial cells and a 3D matrix has in vitro potential for autonomous generation of monocyte-derived DCs and macrophages. Briefly, in a model based on one monolayer of endothelial cells grown to confluency over a 3D collagen membrane, monocytes from total PBMCs selectively extravasate and differentiate into either resident macrophages or migratory DCs with potent antigen-presenting capacity in the collagen matrix.

FIG. 35. Confocal/Hoffmann summation

microscopic image of B (loaded with Cell

Tracker Red) and T (loaded with Cell Tracker green) cells forming aggregate zones in the presence of DCs (unstained).

FIG. 36. A photomicrograph illustrating a cytospin autoradiograph/cytochemistry preparation of an in vitro GC cluster. Cells with silver grains (black) are dividing B cells. Orange stained cells in the center of the cluster stained with FDC-M1 are FDCs.

FIG. 37. The regeneration of an FDC network after culture on collagen film for 30 days. Note the iccosome-sized particles.

FIG. 38. Schematic of the preparation of an MTE model in 96-well plate format.

FIG. 39. Schematic representation of the preparation of an MTE model in 96-well plate format.

FIG. 40. Exploded view of embodiment using dialysis membrane in the construct.

FIG. 41. Schematic of example experimental protocol to examine an immunological response using the artificial immune system of the present invention.

### EXAMPLES

### Example 1

### Designer Scaffold Structures

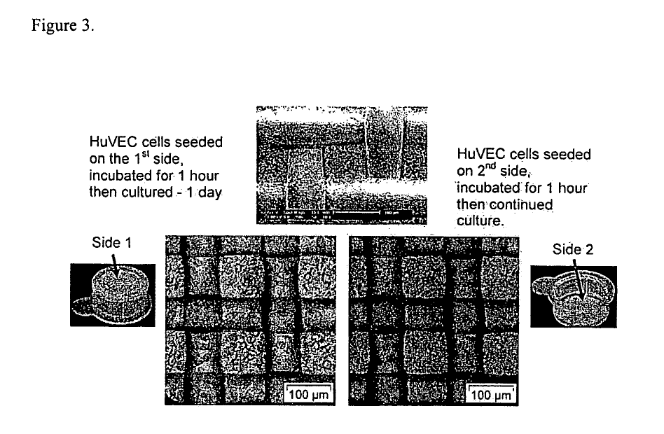

Designer scaffold structures were constructed to test cell viability, cell motility, and nutrient flow for bioreactors and have studied cell motility as a function of construct stability for collagen gels. FIG. 3 shows HUVEC cells growing on protasan/collagen matrix on a nylon mesh. High magnification SEM of the nylon membrane and interspersed Protasan/collagen matrix material is shown in the top image. Seeding of the primary layer of HUVEC cells was accomplished on an inverted membrane (left, Side 1), then 24 hours later, brought to an upright position (right, Side 2) where the second layer was applied. Phase contrast images of each plane of HUVEC cells is shown in the center two lower images, with the left being the first layer, and the right being the second layer applied.

### Example 2

### Digital Printing Technology

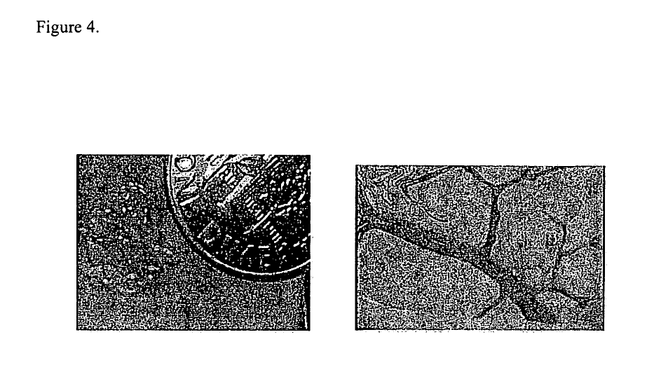

Preliminary hardware and software ETC heterogeneity digital printing prototypes have been developed. FIG. 4 shows the mockup of a digitally printed lymph node and a retinal image of vasculature. This mockup lymph node comprises six biocompatible hydrogel layers, four different patterns, and three materials. The vasculature image has been built with multiple layers of biodegradable construction material with feature sizes that range from about 100 to about 3,000 microns. The objects were fabricated with three dispensing nozzles each.

### Example 3

### LTE Structure

The LTE serves as an important locus for activation of naive T and B cells. The present invention includes, in the design of the LTE, multiple approaches for fabrication of a model of the lymph node extracellular matrix and providing various microenvironemental cues (such as chemokines, cytokines, cells (e.g., fibroblastic reticular cells)). Specific design considerations for the LTE include T cell activation and DC survival/function within the LTE and fabrication of LTE structures comprising both T and B zones. These can be assembled using several complementary strategies.
a. Direct physical assembly of segregated T and B cell areas.
b. Self organization and maintenance of T and B cell areas via creation of engineered local chemokine sources within distinct locations with the matrix.
   The following description sets out in detail the experimental rationale and approach for each of these features of the present invention.

### Example 4

### Microbeads Fabricated from Lymphoid Extracellular Matrix

Microbeads were fabricated from porcine lymphoid extracellular matrix prepared using a protocol provided by Dr. Stephen Badylak, University of Pittsburgh.

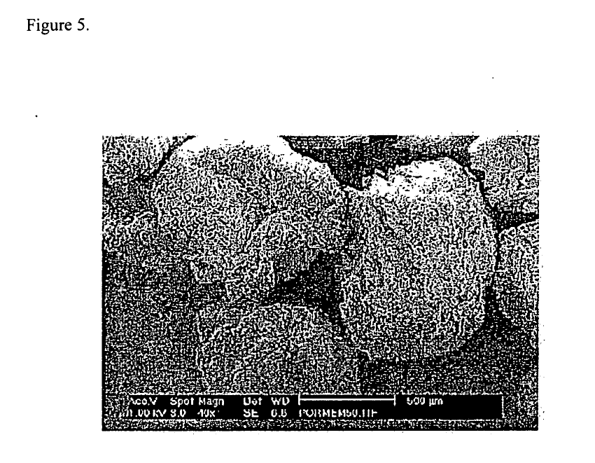

A suspension containing ˜10 mg/ml lymph node (LN) ECM microfragments in 2 mg/ml Protasan, pH 3.5, was sprayed over the surface of liquid nitrogen in a laminar, drop-by-drop mode, making droplets of about 1.5 mm in size. The frozen beads were then freeze dried overnight, incubated in 10% tripolyphosphate (TPP), pH 6.0, for 1 hour thereafter, then washed three times with deionized water over a 100 μm cell strainer, and were then freeze-dried again (FIG. 5).

### Example 5

### In Vitro Tissue Slice Templates

Additional approaches to constructing a functional LTE. The embodiments above describe an approach to fabricating a minimal, functional mimic of mammalian, preferably human, secondary lymphoid tissue. Other embodiments considered within the scope of the present invention are now described.

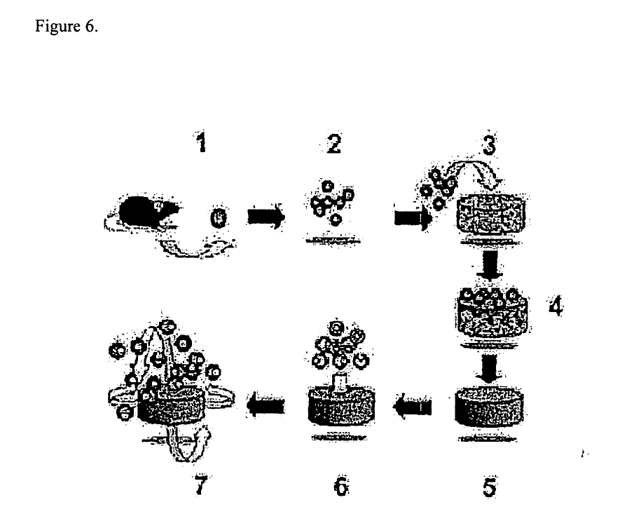

Another embodiment involves ‘templating’ the LTE using native human stromal cells (FIG. 6), in a manner similar to that reported by researchers attempting to develop an in vitro artificial thymus (Poznansky, et al., Nat. Biotechnol. 18:729-734 (2000)). Their approach comprised the following steps:

1. small thymus fragments from mice were cultured on the surface of Cell Foam disks (a porous matrix) in 12-well plates and covered in growth media for 14 days until a confluent layer of stroma had formed throughout the matrix.

2. upon reaching confluence, human lymphocyte progenitor cells were added into the co-culture.

3. during co-culture for 4 to 21 days, non-adherent cells were periodically harvested and cell surface markers were analyzed to determine T lymphopoiesis.

Following a similar scheme, in an embodiment of the present invention, LTE matrices could be “templated” with stromal cells derived from lymph node fragments or lymph node, spleen, or tonsil “slices” to seed the construct with native stromal cells and provide a ready microenvironment for added T cells, B cells, and DCs. Such cocultures can be maintained in vitro using standard organ culture methods during the templating step, and the templated LTE can subsequently be loaded into the AIS bioreactor for continued maintenance. This approach not only provides an alternative for generating a correct lymphoid microenvironment, but also a complementary in vitro approach for analysis of lymph node formation and organizing principles.

### Example 6

### Bioreactor Design and Construction: Integration of the AIS Components

Drawing an analogy with high throughput drug screening technology, an AIS suitable for rapid vaccine or chemical screening can use multiple, low-cost, disposable bioreactors, designed for single-use. Each bioreactor will be challenged with a different antigen and, upon activation of the immune response, harvested for antibodies, B cells, and T cells.

In an embodiment of the present invention, microfluidic bioreactors can be used to achieve this goal. They provide the additional advantage of requiring low numbers of scarce cells for seeding tissue constructs.

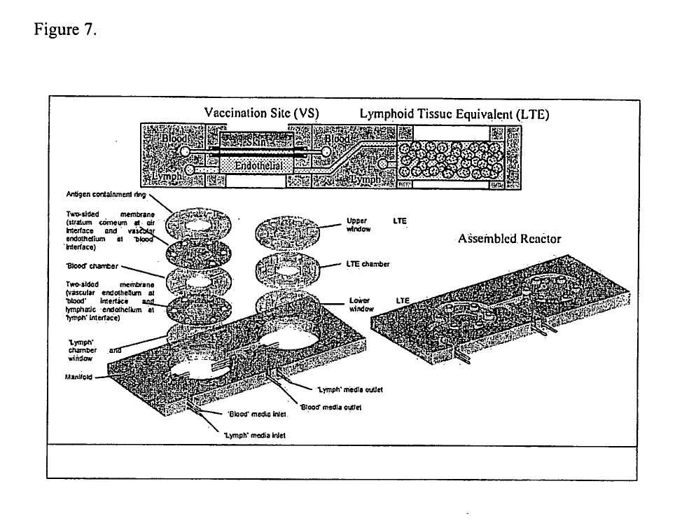

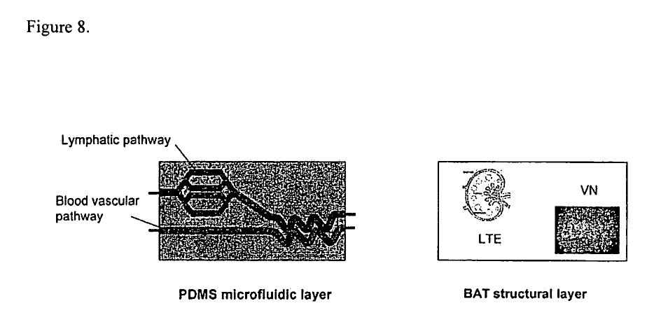

As illustrated in FIG. 7, in an embodiment of the present invention, the AIS bioreactor can be fabricated as a two-compartment microscope slide with a transparent polymer sheet or glass coverslip for microscopic examination. In a preferred embodiment, the physical dimensions of each immune bioreactor measure of the order of about 7.5 cm long and about 2.5 cm wide, with an overall thickness of about 2 mm or less. The first chamber contains the VS and LTE membranes that can be grown as modular units and later inserted into the lower structural layer or as a fully integrated system from the start. The second chamber contains the LTE, comprising T and B cell populations. If required, additional LTE constructs can be added to enable lymphoid organ trafficking or trafficking to other tissues. Syringe tube ports located on the upper layer permit injection of factors and/or cells at strategic positions along the vascular pathways and within ETCs. FIG. 8 shows a plan view of an example integrated bioreactor that shows micromachined pathways with high contact area beneath the VS and LTE ETCs.

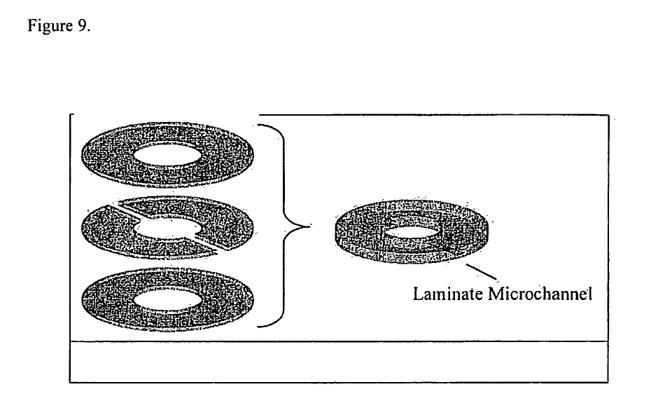

To promote interaction between cells migrating along pathways and in the VS and LTE tissue constructs, the contact spacing between each tissue membrane can be adjusted by using, e.g., machined inserts or thin laminates that have small, integrated microchannels. Suitable construction materials include biologically compatible polymers, such as polycarbonate, polyethylene, and acrylic. A laminate-based insert is as shown in the example (FIG. 9), where as a larger milled tubular design is incorporated in to the design illustrated in FIG. 7. In a sense, these designs mimic a thin venule pathway that supports lymphocyte migration from peripheral blood into secondary lymphoid organs.

Nutrient-rich media can be pumped from an external media reservoir through the channels, flowing tangentially past the VS and LTE constructs, and back to the reservoir. Nutrient and waste product transport between the recirculating media and the tissue constructs occurs through both diffusional and convective (Starling flow) processes.

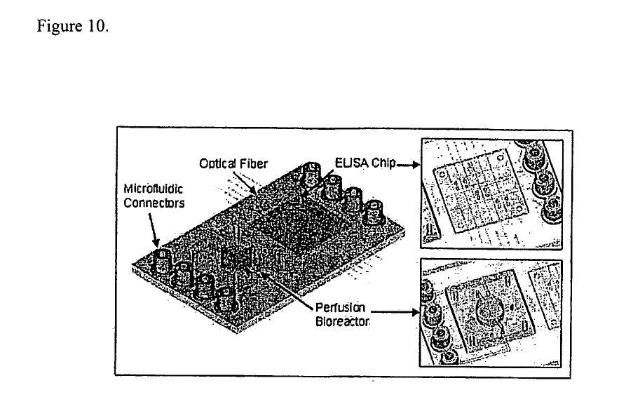

In contrast to other nutrients, oxygen is only sparingly soluble in cell culture media. Consequently, high perfusion rates may be required to sustain a sufficient oxygen supply and to avoid developing necrotic zones. Should required perfusion rates exceed physical capabilities (e.g., unusually high pressure drops can compromise the integrity of bioreactor seals) or generate excessive fluid shear, in alternative embodiments, the oxygen tension in the media may be increased by, for example, using an O₂ microexchanger in-line with the circulating blood media. By circulating the blood media over gas permeable polymers, exposed to high oxygen concentrations on the opposite side, the O₂ environment can be adjusted to compensate for any O₂ consumption and loss. Monitoring and making adjustments to the O₂ concentration in the bioreactor can be accomplished using commercially available non contact fluorescent probes to provide feedback to an oxygen air supply. Creating a high concentration gradient between the gaseous oxygen at the polymer interface and the tissue construct, can facilitate diffusional transport and culturing of thicker constructs. An example of an assembled construct with transparent covers for optical inspection/fluorescent imaging is shown in FIG. 10.

### Example 7

### Fabrication and Assembly of Layered AIS

Fabrication of such microfluidic bioreactors may require ultra short pulse machining trials with the biocompatible materials to determine optimum processing conditions (such as laser fluence and translation speed). The design of the present invention is sufficiently flexible to allow laser machining of a layered device (e.g., gas permeable polymer top layer, BAT deposited middle layer, and PDMS bottom layer) for additions of vias or ports after the device has been assembled.

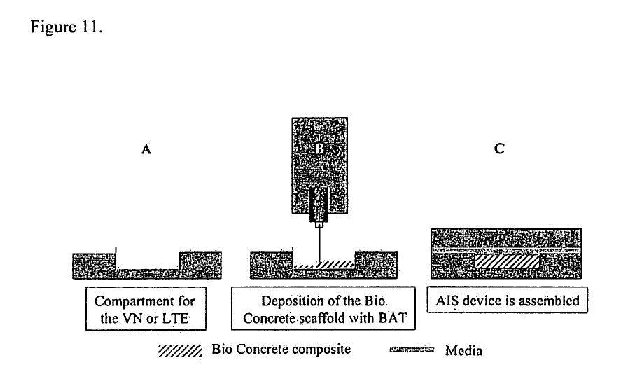

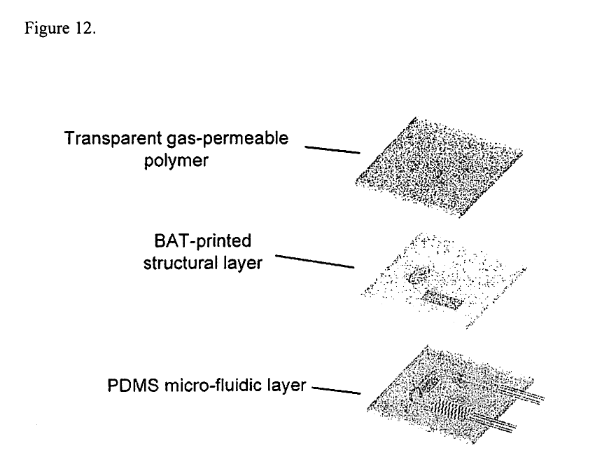

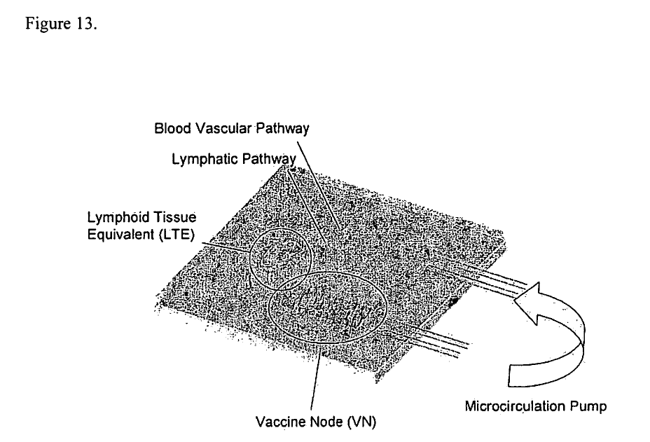

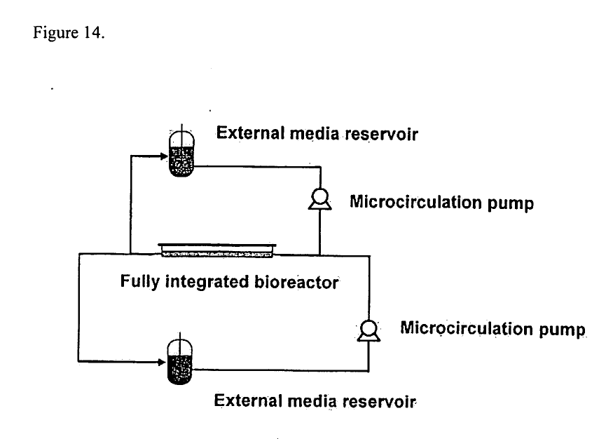

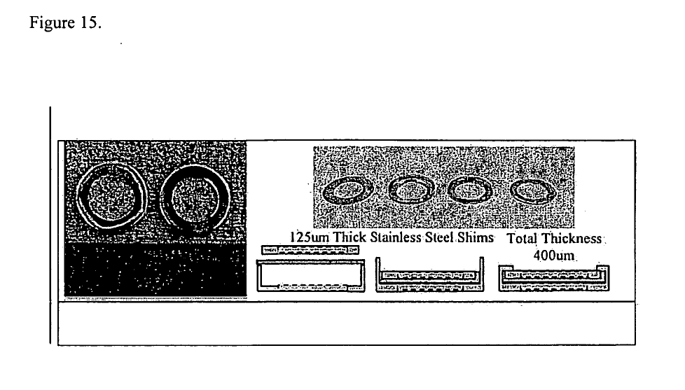

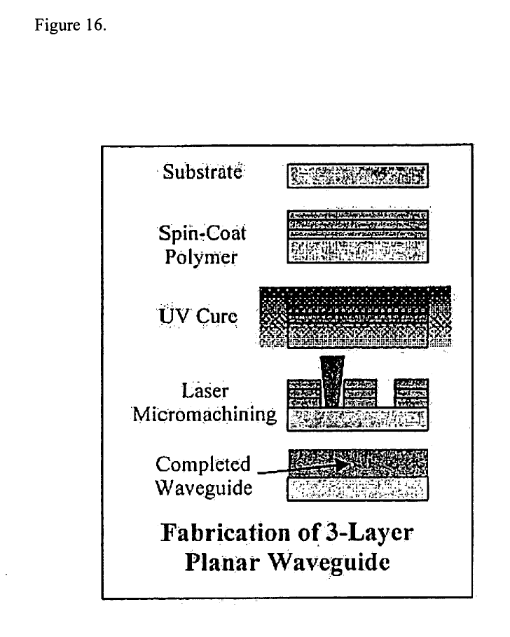

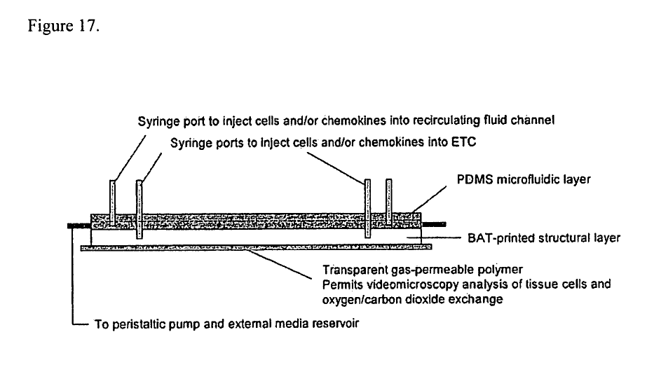

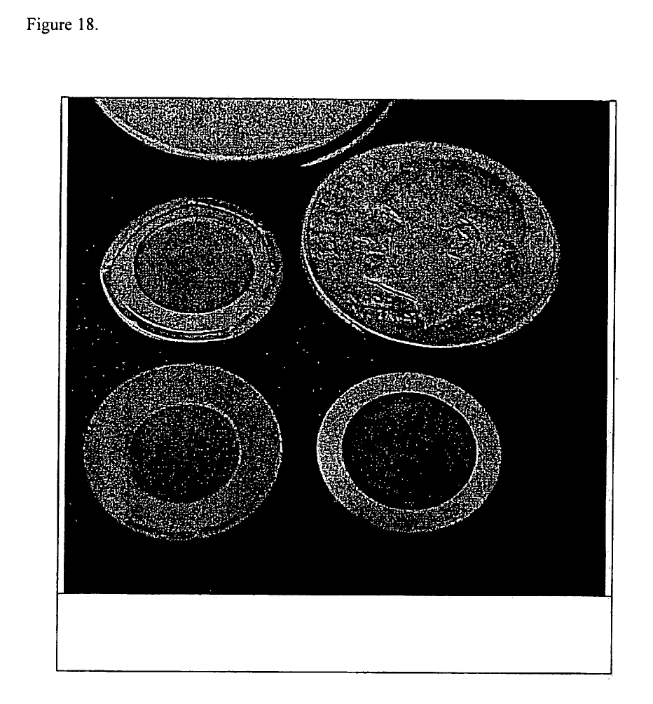

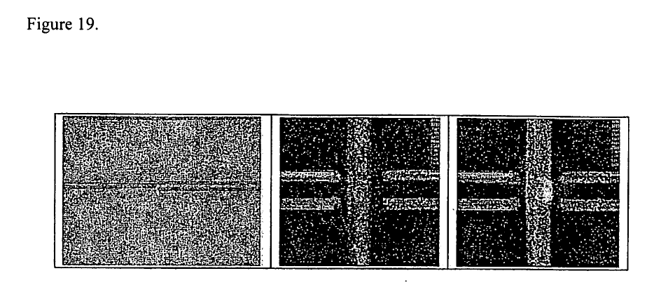

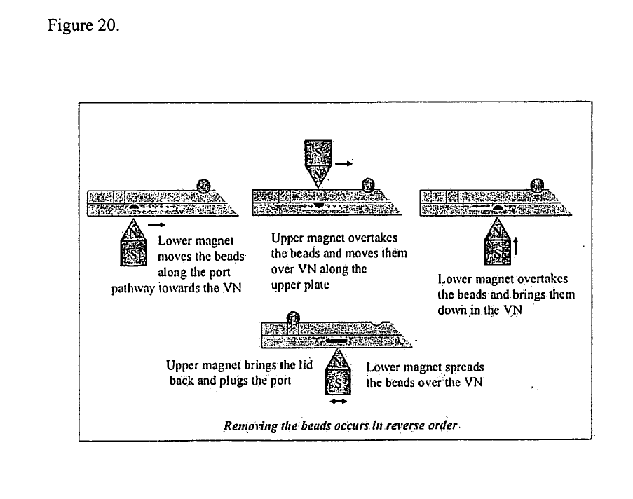

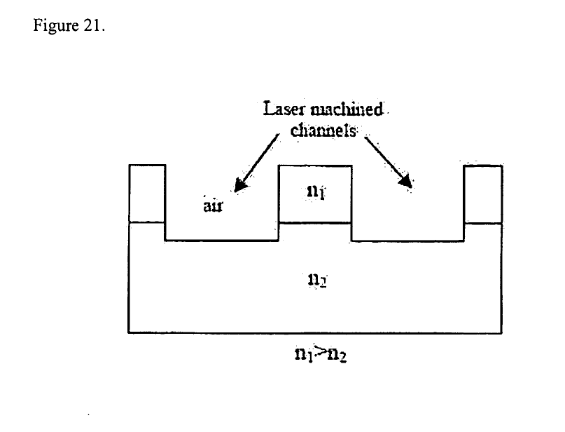

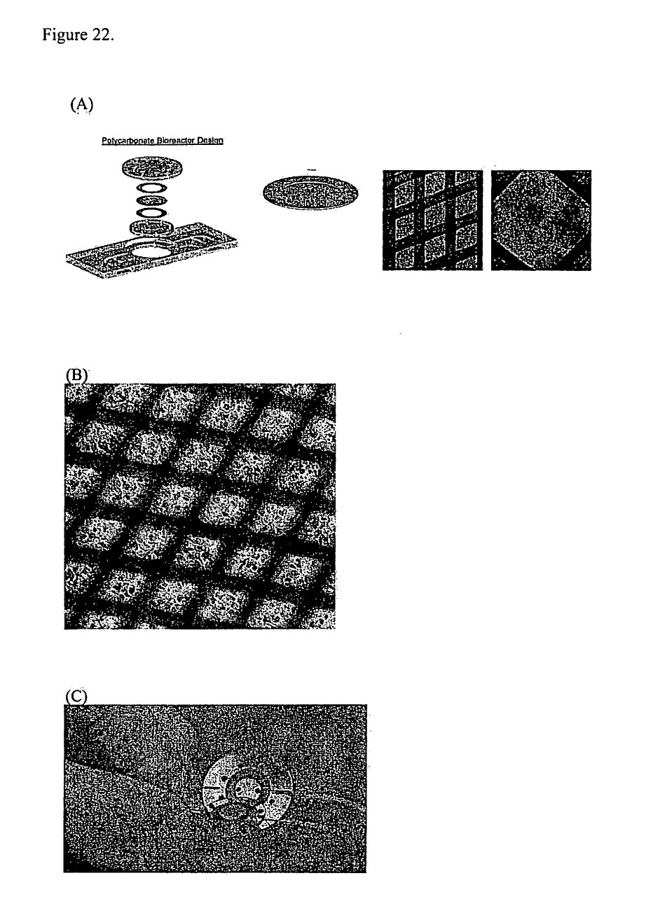

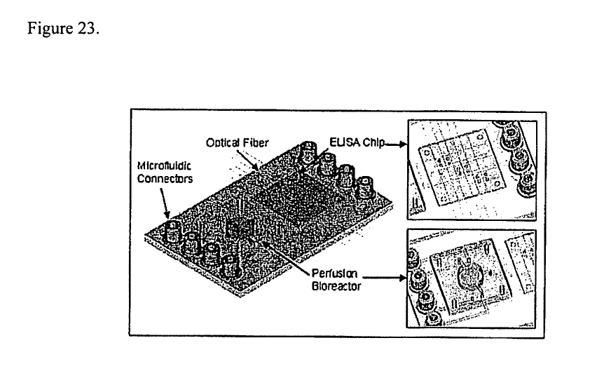

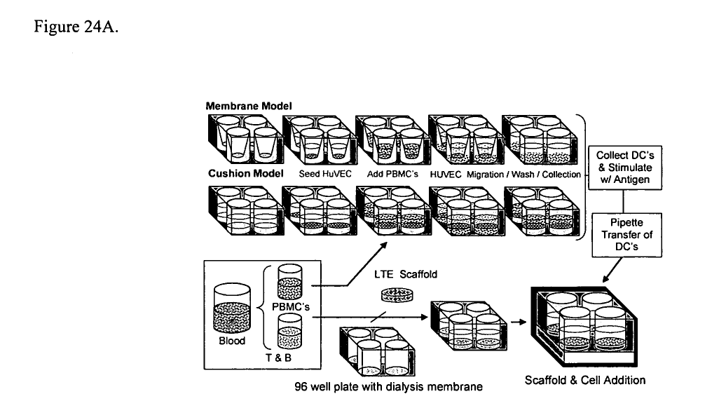

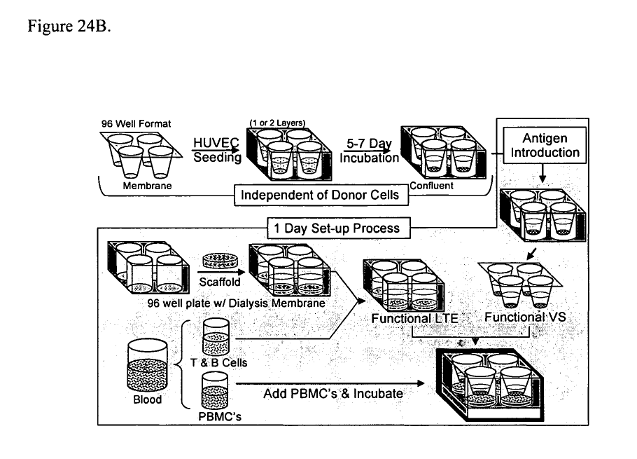

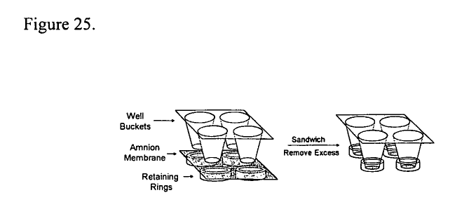

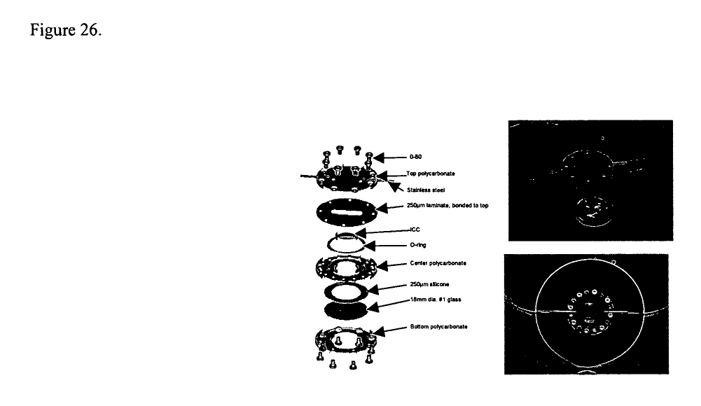

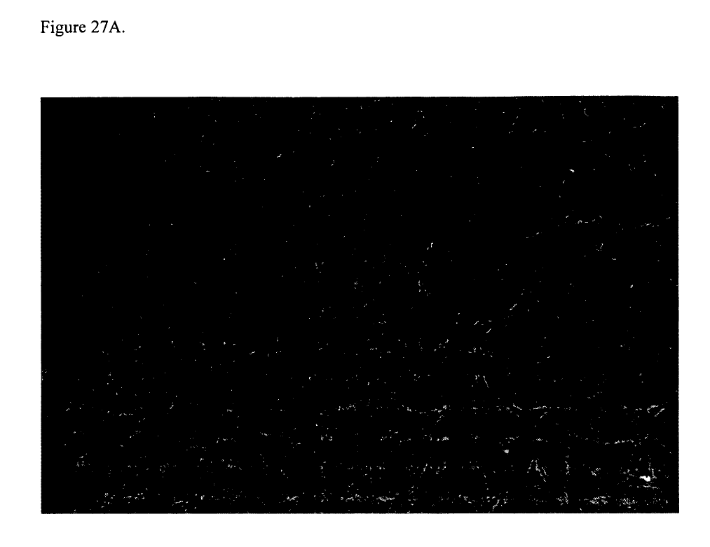

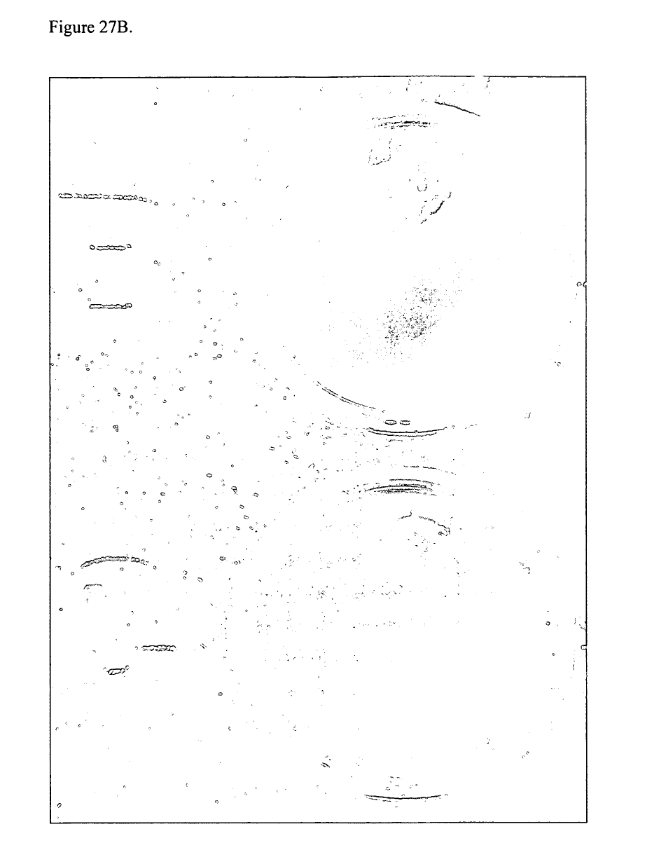

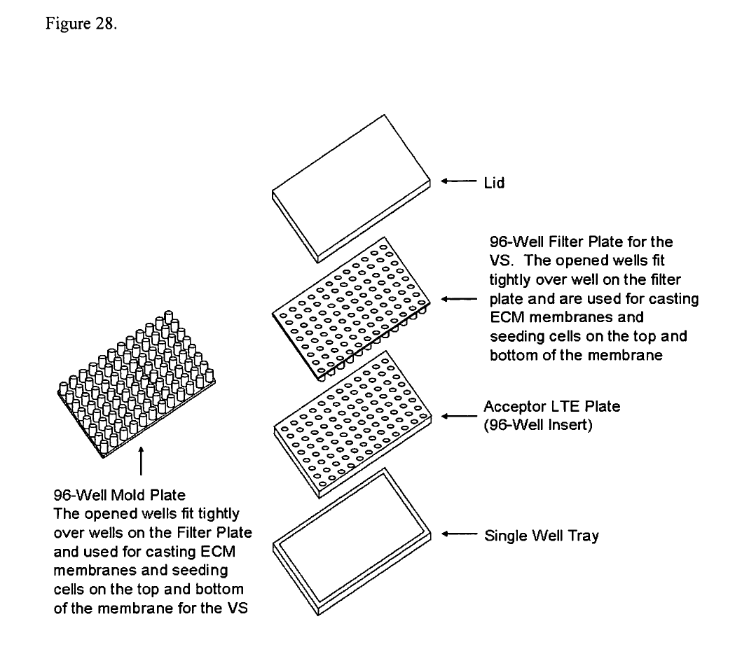

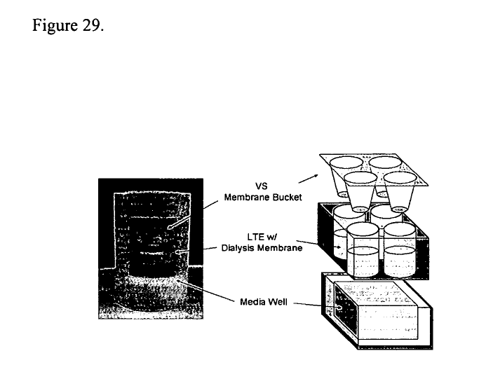

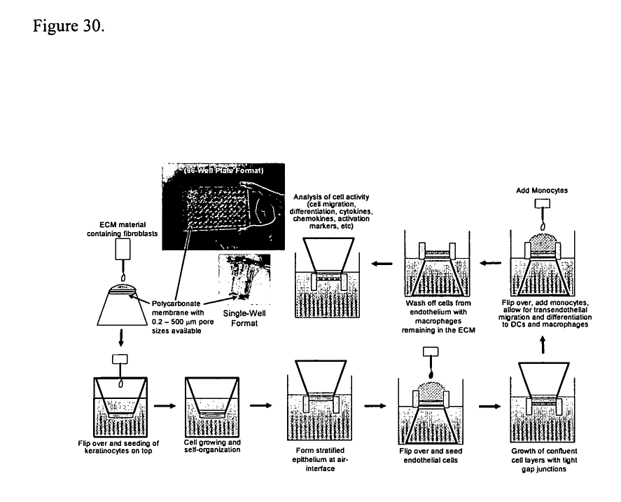

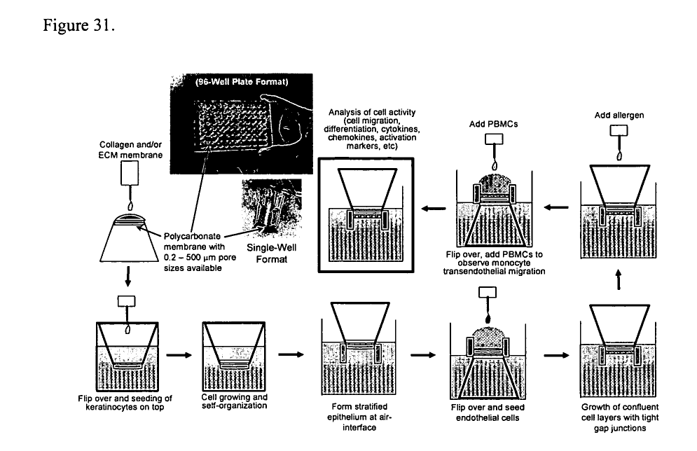

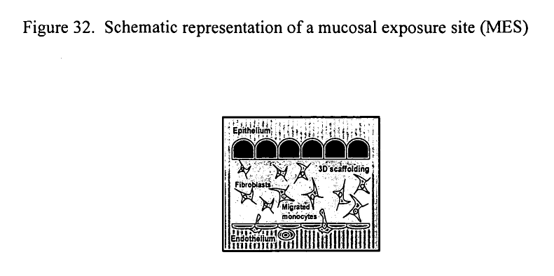

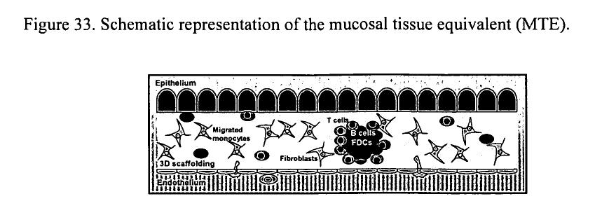

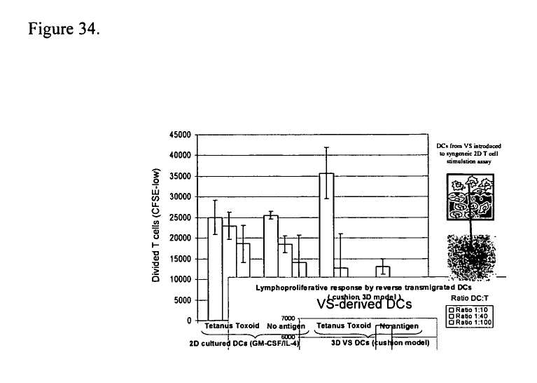

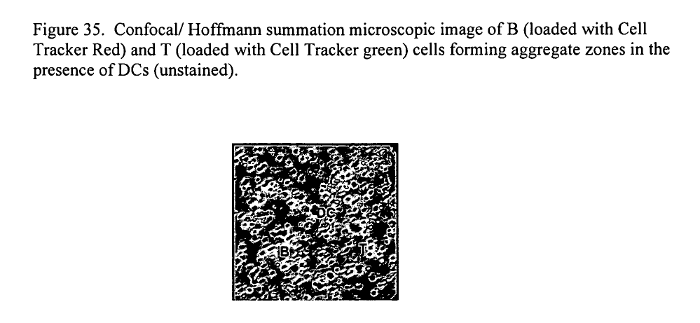

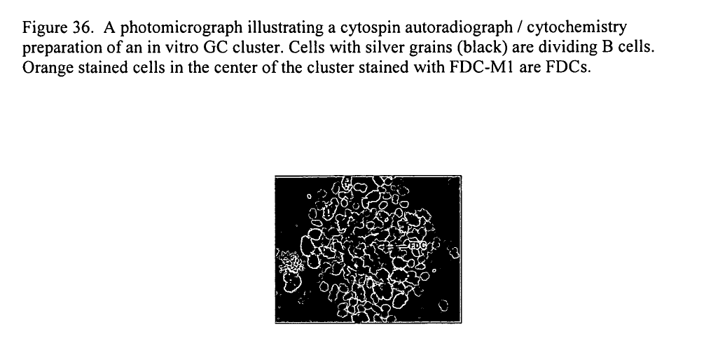

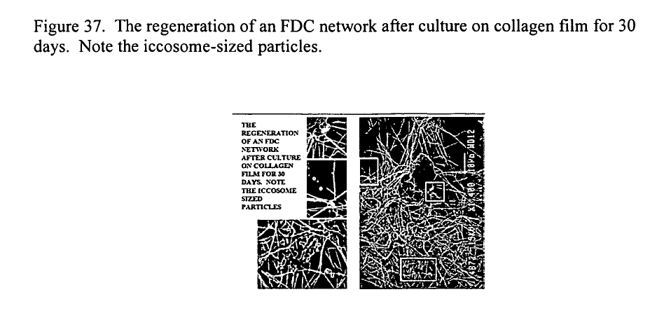

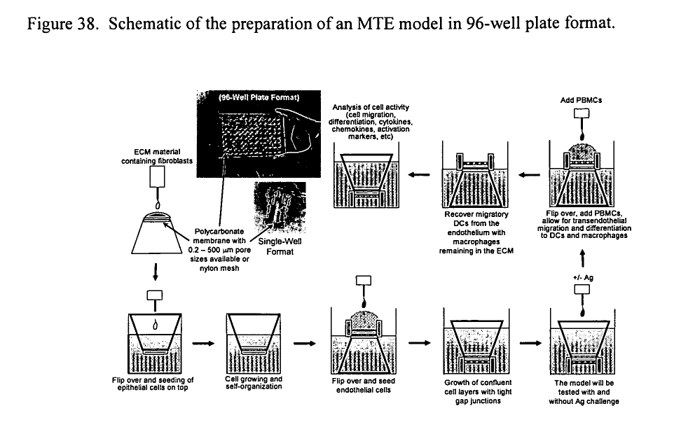

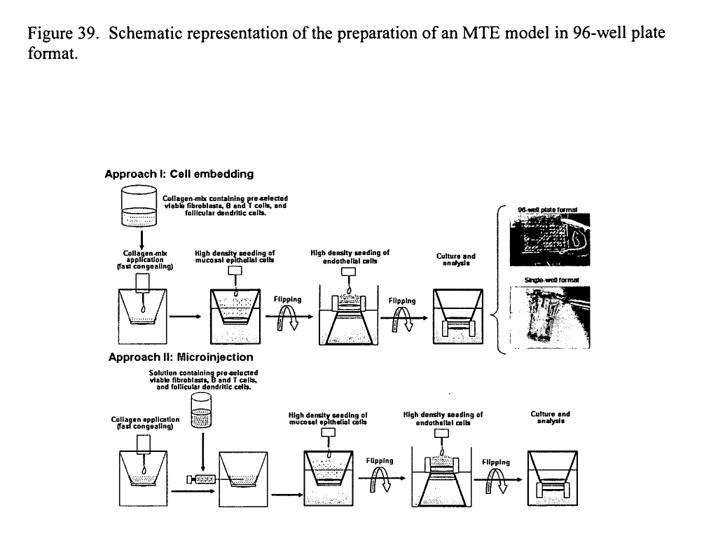

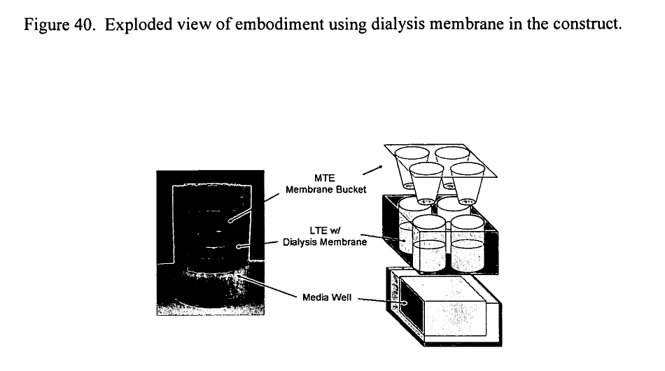

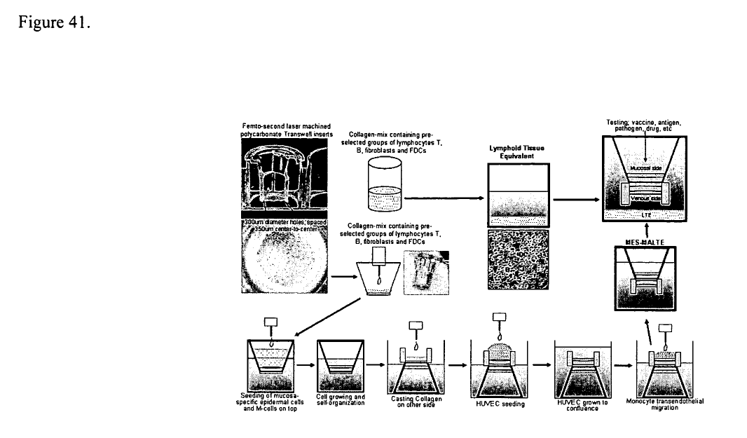

FIG. 11 shows cross sectional views of direct deposition in an embodiment of an AIS device. Various biomaterial structures can be incorporated as constituents of the artificial immune system (e.g., bio concrete, inverse hydrogel opal, colloidal particles, ECM gels, collagen gels, microcarriers). For example, a polymeric mesh rebar can be deposited layer by layer directly in the recessions of the VS and LTE areas. In such a design, it is preferred to have the lower plate of the AIS unit made of polyacrylate, polystyrene, or another transparent plastic sensitive to DM, to allow the mesh rebar to attach to the plate. In this embodiment, the surface will be micro-patterned using KOH in a manner similar to the ESC scaffolds. Fibrin gel matrix bearing all necessary nutrients and cytokines will be used to coat the threads of the mesh as a thin film, leaving sufficient space for cell accommodation and motion.

As shown in FIGS. 12 and 13, the design of the present invention is sufficiently flexible to allow laser machining of a layered device (e.g., gas-permeable polymer top layer, BAT-deposited middle layer, and PDMS bottom layer). FIG. 14 provides a schematic diagram of a perfused bioreactor system with the associated external pumps for the lymphatic and blood vascular loops and external media reservoirs. The AIS bioreactor can be operated in either semi-batch or continuous mode.

In an embodiment of the present invention, integration of membranes in the bioreactor is achieved by crimping the membranes between thin metal (e.g., stainless steel) rings, as illustrated in FIG. 15. Using such a crimping method, biological membranes can be supported without use of adhesives and can be pressed into a disk with thickness profile of about 400 μm or less.

FIG. 16 shows the fabrication of a 3-layer planar waveguide. FIG. 17 shows an example device comprising a perfusion bioreactor, ELISA chip with integrated optical waveguides, microfluidic backplane to connect and allow swapping of devices and microfluidic connectors for external pumps and reservoirs.

In addition to machining channels directly, molds can be machined in suitable materials to create a reusable master from which PDMS devices may be formed. This will allow a higher volume of devices to be fabricated than laser machining in serial. Channel encapsulation methods will be evaluated to provide a leak-proof construct. The materials that comprise the device will likely be damaged at high temperatures, so robust, low-temperature bonding methods will be needed.

Testing of the devices will require fixtures for mounting and providing external connections. Laser machining can also be used to provide manifolds for these test fixtures that would support fast swapping of devices without the need to disconnect external pumps or reservoirs. Equipment for measuring pressure, flow resistance and flow rate can also be connected to the devices via the manifold. Revisions to optimize the channel geometries can be made based on this data and performance of the ETCs.

An AIS microfluidic bioreactor system can be placed in an incubator that maintains constant temperature, humidity, and carbon dioxide control. Phenol red can serve as a colorimetric pH indicator in the media, so that pH can be monitored, e.g., periodically through visual inspection or photometric determination with logging capabilities. In another embodiment, pH can be monitored continuously and precisely in the external media reservoir with a pH probe and recorder.

Creating insert supports for both synthetic and natural membranes has been accomplished by using laminates, crimped rings, and adhesives (FIG. 18). Laminates and adhesives have primarily been used to support polymer meshes, which in turn are provide mechanical strength to synthetically formulated biological membranes. Fabrication using the laminate comprises sandwiching a stretched mesh between two pieces of polymer laminates, which are then thermally sealed together. The adhesive method comprises stretching a mesh support and adhering a stainless steel ring using a biocompatible glue. The crimping method, discussed earlier, comprises compressing the membrane between two stainless steel rings. Generally, the laminate and adhesive methods are limited to synthetic mesh-supported membranes, while the crimping method can accommodate both natural biological membranes and synthetic meshes.

### Example 8

### Optically Diagnostic AIS Microfluidic Bioreactor

Immunology has many cascades of events that cannot be observed in any human system at this time. In particular, if a vaccine fails as a result of a rate-limiting step related to entry into and interactions within an immunological tissue, there is presently no method to measure or improve this process in humans. To address this problem, an embodiment of the present invention include building the AIS in such a way as to be able to optically monitor in situ the steps of the in vitro immunological/vaccination process.

In one embodiment, integrated optical waveguides become part of a micro-total analytical system (μTAS) of the AIS, with many different functions including optical excitation, absorption, fluorescence, and imaging on a single microfluidic bioreactor system. An in situ diagnostic system will make optimization and conducting diagnostic evaluations of the immunological constructs more rapid. Two-photon fluorescence can enable visualization of immunological events in all three dimensions in both artificial and living tissues. This technique can aid in understanding and optimizing the effects of various adjuvants, vaccine candidates, drugs, biologics, biomolecules, and antigen presentation vehicles in vitro and with in situ diagnostics.

Prototype results are presented regarding fabrication of μTAS that can be used to perform the immunological analysis steps in situ, to simplify the process and reduce analysis time. In one embodiment, the present invention provides an AIS device with the addition of integrated optical waveguides for in situ optical diagnostics. These waveguides provide optical excitation and detection pathways for calorimetric analyses (such as ELISA assays, absorption and fluorescence analysis).

In this example, single layer, planar polymer waveguides were fabricated using selective femtosecond laser ablation of a polymer substrate. A glass slide was coated with an 80 μm-thick layer of a single part, ultraviolet curing polymer with a refractive index of 1.56. After curing for 30 minutes with a ultraviolet (UV) lamp (4W), planar optical waveguides and microfluidic channels were machined into the polymer using a Ti:sapphire femtosecond regime laser. The optical waveguides and microfluidic channels were each approximately 100 μm wide by 80 μm deep. Light from a CW Nd:YVO₄ laser was coupled to the planar waveguides through a 50 μm core diameter optical fiber inserted into a tapered alignment groove as shown on the left. Light guided through the planar waveguides passes through an intersecting microfluidic channel. This waveguide/channel intersection is shown in the middle with the laser source off and on the right with the laser source on. Light entering the channel from the right is collected in the waveguide on the opposite side of the channel. This light is then coupled to another 50 μm core optical fiber and sent to a silicon detector for measurement.

### Example 9

### In Situ Diagnostic Bioreactor Development

Microfluidic devices that mimic in vivo systems are proving valuable in studying cell interactions and biological processes in vitro. Such devices offer several advantages over traditional large-scale fluidic assemblies including small sample and reagent volumes, small waste volumes, increased surface area-to-volume ratios, low Reynold's numbers (laminar flow), fast sedimentation for particle separation, reduced reaction times, and portability. Some microfluidic devices also integrate pumps, valves, filters, mixers, electrodes, and detectors. The ease of alignment and shorter reaction times make near real-time detection possible using this approach.

Fabrication of microfluidic devices has relied mainly on technology developed in the microelectronics industry, such as photolithography and subsequent etching of silicon or glass. These technologies often require multiple processing steps and clean room facilities and can take days or weeks to produce a working device; they are better suited to mass production of devices than rapid prototyping. A relatively new method of fabrication is ultra-short pulse laser micromachining (USPLM). USPLM has the advantage that materials can be machined directly without the need for masks or photoresist development. Devices can therefore be fabricated more quickly, often in a day or less, permitting rapid prototyping. Furthermore, due to the extremely short pulse duration (<150 fs) and high intensities, almost any material can be readily ablated because of multiphoton absorption and ionization, even if it is transparent at the laser wavelength. This is especially useful in machining materials for an optically transparent bioreactor. FIG. 19 shows an ultra-short pulse laser micromachined planar optical waveguides integrated into microfluidic channel. Left panel: Tapered port for fiber optic coupling. Middle panel: microfluidic channel intersection of planar waveguide (source off). Right panel: microfluidic channel intersection of planar waveguide (source on, entering from right).

In an embodiment of the present invention, USPLM was used to machine microfluidic channels, vias, reservoirs, and integrated optical waveguides in the bioreactors. An inexpensive and widely used biocompatible silicone elastomer, polydimethylsiloxane (PDMS), comprises the main body of the structure. Sheets of PDMS can be patterned by USPLM and then assembled to form the 3D construct (Laser-machined microfluidic bioreactors with printed scaffolds and integrated optical waveguides, Nguyen, et al., Proc. SPIE Int. Soc. Opt. Eng., 5591). The layers may be either permanently bonded by treating with oxygen plasma or temporarily bonded by applying mechanical pressure. Thus, fabrication of disposable or re-usable devices is easily accomplished

In one embodiment, integrated optical waveguides are fabricated as illustrated in FIG. 20. The waveguides comprise multiple alternating refractive index polymer layers in which the middle polymer layer has the higher refractive index. In preferred embodiments, the polymers can be either UV or thermal cured or a combination of both (e.g., PDMS cladding and UV curing core). The waveguides are defined by removing material on either side using an ultra-short pulse laser. The laser can also be used to integrate tapers for fiber optic coupling to the waveguides. Microfluidic channels are machined either parallel or perpendicular to the waveguides. Light is launched into a waveguide on one side of the microfluidic channel, passed through the channel where it interacts with the fluid in the channel and then collected by the waveguide on the opposite side of the channel and sent to a detector. In another embodiment, fiber optics are embedded into PDMS and then microfluidic channels machined perpendicular to the fibers, removing a small section of the fiber in the channel. This eliminates the need for planar polymer waveguides and fiber-to-waveguide coupling losses at the expense of elaborate waveguide geometries, such as splitters and combiners FIG. 21.

FIG. 22 shows an example bioreactor construction with collagen membranes on rings and support matrix. Collagen cushion congealed at 37° C. for 1 hour remained highly stable with no collagen degradation for more than 3 weeks. Panel A shows the bioreactor design. Panel B shows progression from the whole bioreactor to the level of the collagen matrix cushion within the mesh. After the HUVEC cells have reached confluence on the collagen cushion, the bioreactor is assembled under sterile conditions (Panel C). Once assembled, media flow is initiated.

### Example 10

### Design of an AIS Device

An example AIS device is illustrated in FIG. 23. The device comprises a microfluidic bioreactor, ELISA chip with integrated optical waveguides, microfluidic backplane to connect and allow swapping of devices and microfluidic connectors for external pumps and reservoirs. The bioreactor has four external ports, two each above and below the tissue construct. An ELISA chip with three sets of two channels is illustrated, though more channels are contemplated in the same footprint in other embodiments. In each set, one channel is for a sample assay and the other is a control with no sample. Each set is attached to the same ELISA input port, allowing both channels to be prepared simultaneously; however, only one channel in a set is attached to the sample fluid. This fluid is pumped from the bioreactor to the ELISA chip through a channel in the microfluidic backplane. Valves control the addition of the sample fluid to each channel. Light is coupled to the ELISA channels through optical fibers and the transmitted light is coupled to another fiber attached to a detector. In this preferred embodiment, the bioreactor and ELISA chips are both optically transparent for two-photon and confocal microscopic examination. In this preferred embodiment, the footprint of the entire assembly in this example is approximately 50×75 mm.

### Example 11

### Utilizing AIS as a Biofactory

In an embodiment of the present invention, the assembled LTE is used as a “biofactory,” biosynthesizing various desired biomolecules (such as cytokines, proteins, antibodies). For example, if an antigen is presented to B cells, they can create antibodies in the LTE. Potentially, the created antibodies could also be monoclonal, depending on the repertoire of B cells and how the peptide is presented to the B cells. Monoclonal antibodies (mAb) are used extensively in basic biomedical research, in diagnosis of disease, and in treatment of illnesses, such as infections and cancer. Antibodies are important tools used by many investigators in their research and have led to many medical advances.

### Example 12

### Static AIS

Drawing an analogy with high-throughput drug screening technology, an AIS suitable for rapid vaccine, vaccine formulation, or chemical screening can use multiple, low-cost, disposable bioreactors, designed for single-use. Each bioreactor will be challenged with, for example, a different antigen or antigen/adjuvant combination, and, upon activation of the immune response, harvested for antibodies, B cells, and T cells. An embodiment of the present invention is illustrated in FIG. 24. In this example, a static 96-well plate format is used. The system comprises two parts: the MTE and LTE. Each part of the system can be treated separately and then they are combined subsequently. The 96-well format can accommodate, e.g., amnion membrane and collagen MTE models as well as various LTE designs (e.g., tennis ball model and inverse opal scaffolds).

### Example 13

### Integrated AIS

Drawing an analogy with high throughput drug screening technology, an AIS suitable for rapid vaccine or chemical screening can use multiple, low-cost, disposable bioreactors, designed for single-use. Each bioreactor will be challenged with a different antigen and, upon activation of the immune response, harvested for antibodies, B cells, and T cells. In another embodiment of the present invention, an integrated AIS comprises a construct to which PBMCs are added (FIG. 24B). The preparation of the MTE and LTE are similar to that described for the static model, but in the MTE, antigen is incorporated in the membrane before the addition of PBMCs and after the HUVECs have reached confluency.

### Example 14

### Dialysis Membrane Integration

In further embodiments of the present invention, dialysis membranes can be incorporated in the design of the AIS to reduce the need for media exchanges, which can improve cell viability and improve the detection of low concentration molecules, including proteins and antibodies.

By using dialysis membranes in the LTE, the incubation well can be designed to allow small molecules to pass freely across the membrane while larger molecules, such as proteins, antibodies, and cytokines, can be retained. The permeability to small molecules provides a means of removing cellular waste, thereby keeping cells viable for longer periods, while the retention of large molecules in each of the localized wells can increase the probability of cytokine or antibody detection.

Cell viability. Assessment of the ability of dialysis membranes to increase cell viability was conducted by preparing cell cultures with and without a dialysis membrane. Cultures of 1 million PBMCs were added to 500 μl of media and were stimulated with PMA and PHA. Each culture was then placed in either a normal 96-well plate or in a dialysis membrane holder (with 3.5 kDa cut off cellulose membrane) suspended in an additional 5 mL of media. A comparison well with 1 million PBMCs in 5.5 mL was prepared as a standard. The cells were then incubated for 3 days at 37° C./5% CO₂. After 3 days, the cultures were removed and inspected (visually) for pH changes. The medium in the ‘normal’ well had turned yellow, indicating acidification and that conditions were not conducive to continued cell growth. The medium in the dialysis membranes-containing culture vessels remained pink, indicating a slightly basic pH, optimal for continued cell growth.

Large molecule retention. Assessment of the ability of dialysis membranes to retain large molecules was conducted by monitoring whether a 50 kDa albumin molecule could permeate across a 10 kDa cut off dialysis membrane. A stock solution of albumin (5 mg/mL) and 1% NaCl was prepared and placed in an open well plate. The 10 kDa dialysis membrane ‘bucket’ was then suspended in the plate and 500 μl 1% NaCl was added. The well plate was then incubated for 24 hours at 37° C. The plate was then removed and the dialysis well solution was analyzed using a UV-visible spectrophotometer at a wavelength of 278 nm. Spectral results and a calibration curves revealed that there was no detectable permeation of the albumin across the dialysis membrane.

### Example 15

### Microfluidic Bioreactor

In an embodiment of the present invention a “thin-sheet membrane bioreactor” was prepared. This embodiment comprises a microfluidic bioreactor to house an, e.g., ECM-derived membrane as a support scaffold for the MTE. In an embodiment of the present invention, the ECM bioreactor, the ECM membrane is held in place by two concentric rings: an inner (e.g., PTFE, Teflon) ring and a larger (e.g., polycarbonate) outer ring. The ECM-derived membrane is sandwiched in the narrow (about 100 μm) gap between the two rings by pressing the inner ring into the outer ring, thereby stretching the ECM-derived membrane tight across the opening in the inner ring. A confluent endothelium can then be grown on either or both sides of the exposed ECM membrane. This approach is readily adaptable to a well-based format. In other embodiments, ported lids and/or retaining rings can be attached independently to either side of the ECM/ring structure, allowing for several different experimental configurations. For example, a ported lid on the top side could provide shear to the endothelium while a retaining ring on the bottom would keep the endothelium in a static condition. The lids can be transparent, allowing microscopic inspection of the vaccination site.

ECM membrane for the VS in a well-based format. In this embodiment of the present invention, the method of mounting the ECM membrane using concentric rings, described previously, can be used in a well-based format, as shown in FIG. 25. Here, the inner Teflon ring is replaced with conventional well buckets. The ECM is placed between the buckets and outer retaining rings and the buckets are pressed into the retaining rings, thereby sandwiching the ECM membrane in place. Excess ECM membrane can then be removed, leaving a tightly stretched membrane across the bottom of the bucket on which to grow the cells of the VS. The buckets can be placed in well plates containing media for cell culture.

Scaffold Bioreactor. In another embodiment of the present invention, the microfluidic bioreactor described is modified to house a scaffold. An embodiment of the present invention, the ICC bioreactor, is illustrated in FIG. 26. The design enables ease of assembly and robust sealing. As an example, it houses a 9 mm diameter, 1/16″-thick ICC scaffold. Flow can be applied to one side of the scaffold through a ported window and confined to a thin (250 μm) chamber. The other side of the scaffold is mounted against a thin glass cover slip to allow high resolution microscopic examination. A microscope adapter plate (lower right figure) was also fabricated.

### Example 16

### Integration of Scaffolds in a 96-Well Format

In this embodiment, scaffolds for the LTE have been integrated in a 96-well format.

FIG. 27A, first image, magnification ˜×20. An ICC scaffold is placed in a well of the 96-well plate, in 500 μl water; bottom view (invertoscope), but other scaffolds can be used, including collagen and microcarriers.

FIG. 27B, second image. Top view: well “B” contains 500 μl water; well “C” contains an ICC scaffold in 500 μl water. In this example, the scaffolds are ˜7 mm across, ˜200 μm thick. The cavities are ˜40 μm.

### Example 17

### Well-Based Format of VS and LTE Integration

In this embodiment, a well-based AIS is designed to be used as an in vitro screening model for, e.g., toxins, pathogens, vaccines, and drug evaluations. FIG. 28 shows how the MTE and LTE constructs can be integrated into a well-based format in which the MTE is used in a filter plate and the LTE is placed into the acceptor wells. The MTE fits over the LTE in the design illustrated.

### Example 18

### High-Throughput Testing

High-throughput testing using the integrated AIS can be accomplished using a static 96-well format, illustrated in FIG. 29. The AIS in this embodiment comprises two parts, the MTE and LTE. Each part is prepared separately and combined in the final step of testing. The simplicity of the system enables automation. Furthermore, the 96-well format, or other well-based format, typically used in laboratory automation can accommodate these embodiments of the AIS.

### Example 19

### Preparation of Tissue Constructs

Preparation of heterogeneous tissue constructs with the addition of cells on the top and bottom of the tissue construct to create endothelium and epithelium. A representation of the development of the MTE model used as a mucosal equivalent and how it can be tested with an allergen is shown FIG. 30. In this embodiment, a polycarbonate membrane support structure is used to prepare a 3D ECM membrane comprising collagen, other natural polymers, or synthetic materials such as hydrogels, or combinations thereof.

Once an ECM is established that can structurally support two cell layers, a layer of epithelial cells, such as mucosal epithelial cells, can be grown on one side of the matrix. After the keratinocytes have established and begin to form stratified layers, the cells are exposed to an air interface for continued stratification and formation of tight cell junctions. Once a keratinized cell layer is formed, the construct is inverted and a layer of endothelial cells, such as HUVECs, can be grown on the other side.

Once the endothelial cell layer is established, the construct can be inverted again to reinstate the air interface for the keratinocytes. Once the endothelial cells form a confluent monolayer, the tissue construct is complete and ready for characterization and testing of, e.g., chemicals, cosmetics, adjuvants, antigens, and/or inflammatory signals.

### Example 20

### Introduction of Other Cells

Introduction of ancillary cells inside the 3D construct (FIG. 31). In embodiments of the present invention, fibroblasts or other ancillary cells can be added. Fibroblasts can be mixed with the ECM material before it is added to the membrane support and before the growth of epithelial and/or endothelial cells on the matrix. In embodiments of the MTE, purified monocytes can be added to the endothelium; the cells can then transmigrate into the construct. After the monocytes have differentiated to either DCs and reverse-transmigrated from the construct or to macrophages and remained in the construct, remaining cells can be removed from the surface of the endothelium, and the resident macrophages will remain within the construct.

### Example 21

Tissue constructs based on endothelial cells and a 3D matrix have shown the in vitro potential for autonomous generation of monocyte-derived DCs and macrophages. Briefly, in a model based on one monolayer of endothelial cells grown to confluency over a 3D collagen membrane, monocytes from total PBMCs selectively extravasate and differentiate into either resident macrophages or migratory DCs with potent antigen-presenting capacity in the collagen matrix. This DC differentiation process occurs within 2 days of entering the collagen cushion, similar to published in vivo human data (Newberry & Lorenz (2005) Immunol Rev 206, 6-21).

We also have found that these immature DCs can acquire and process antigen, maturing into potent DCs capable of initiating antigen-specific primary and secondary immune responses in autologous mixed leukocyte reactions (as seen using, e.g., ovalbumin, tetanus toxoid, zymosan). These DCs have the capacity to induce T cell proliferation (as assessed by CFSE-dilution assay, FIG. 42), cytotoxicity responses (CTL assay), cytokine production (IFN-γ, IL-2, and IL-4 by intracellular staining), and induce high T and B cell motility and survival. Furthermore, these MTE-derived DCs are able to pick up weaker antigen signals than 2D counterparts as shown in FIG. 34 (right) using tetanus toxoid as the antigen. Antigen-specific DC maturation has been assessed by expression of surface markers, such as CD1a, HLA-DR, CD83, CD86, and CCR7.

### Example 22

Using this collagen matrix model, we have generated results that suggest the maturation state of the DCs may impact their behavior in the lymph node. Immature DCs/macrophages in the collagen cushion with naïve T cells tend to segregate the T cells into “zones” or clusters (FIG. 35). An explanation may be that local chemokines released from these APCs tend to act like “chemorepellants,” helping to organize the T/B cell zones in a 3D matrix similar to what is seen in lymph nodes. Mature DCs in the collagen cushion with naïve T cells activate these T cells to proliferate and secrete cytokines. Thus, the state of APC differentiation in the model lymph node appears to assist in the formation of the lymph node architecture, or activation of lymphocytes.

### Example 23

Germinal centers (GCs) in vivo are characterized by the presence of FDCs, memory B cells, helper T cells, macrophages, and GC DCs. GCs contain proliferating B cells that produce memory B cells and pre-plasma cells. During a GC reaction there is class switch recombination and somatic hypermutation in the antibodies expressed by GC B cells. Affinity maturation also takes place in the GC. We have shown that our in vitro co-cultures have many of these characteristics. In previously reported murine in vitro GCs, immunoglobulin class switching, somatic hypermutation, selection of high affinity B cells, and affinity maturation were demonstrated.

An in vitro GC has FDCs, memory B cells, and helper T cells as illustrated in FIG. 36. The FDCs and T and B cells naturally self assemble (cluster) together. The model system was studied in 2D culture plates; in embodiments of the present invention, the FDCs are placed in an engineered tissue construct, such as a collagen cushion, where the GCs develop in 3D. FDCs in the in vivo environment are attached to collagen fibers and do not circulate, as most immune system cells do. In an embodiment of the present invention, the FDCs are ‘fixed’ in a collagen matrix to mimic this. Immobile FDCs form a center and the chemokines they secrete acts to define the basic features of an active follicle. When the FDCs are integrated with collagen, they appear to make dendritic processes, something not seen before with FDCs in vitro (FIG. 38). It appears that attachment to collagen makes is important in this; stimulating FDCs with cytokines, antibodies (e.g., anti-CD40), adding B cells and T cells to stimulate the FDCs failed to cause FDCs to make such processes. We have also seen improvement of FDC accessory function on collagen type-I when B cells are stimulated with LPS, a polyclonal B cell activator. FDCs attached to DCs attached to collagen were 3 times as active in promoting antibody production when compared with those floating on plastic plates. These results demonstrate that putting FDCs on collagen enhances their biological activity.

### Example 24

FIG. 41 shows a schematic representation of an example MES model of the present invention and how it can be tested with antigens. It comprises a biocompatible membrane or mesh support structure to prepare a 3D extracellular matrix (ECM) membrane comprising, e.g., collagen or synthetic materials such as hydrogels, or combinations thereof. To add fibroblasts to the model, the fibroblasts can be mixed in the ECM material before it is added to the membrane or mesh support and before the epithelial and endothelial cells are grown on the matrix. It is important to determine the optimal density at which to seed the fibroblasts to provide ancillary support without overgrowing the matrix. When an ECM is established, a layer of bronchial respiratory epithelial cells is grown on one side of the matrix. After these respiratory epithelial cells are established, the construct can be inverted to apply a layer of human-derived vascular endothelial cells to the other side of the ECM (e.g., HUVECs or human pulmonary microvascular endothelial cells (HPMEC) from lung). When the endothelial cells are established, the construct is inverted again and the construct is cultured until the epithelial and endothelial cells have formed confluent monolayers. At this point, the tissue construct is ready for characterization and testing with, for example, antigen, adjuvant, immune modifiers, or inflammatory signals.

### Example 25

To detect whether an antigen challenge causes an adaptive immune response in the MES, the antigen presenting capacity of the migratory cells from the MES is assessed, e.g., in a 2D T-cell stimulation assay. To perform such an antigen challenge, the antigen of interest is applied to the epithelium (FIG. 41). After application of the antigen, the model is inverted and PBMCs containing monocytes and DC precursors that will migrate into the MES and differentiate into migratory DCs and resident macrophages, both of which will be exposed to and process the antigen, are added. The antigen-primed DCs that migrate from the MES are then collected and added to a T-cell stimulation assay comprising CFSE-labeled, autologous, negatively-selected CD3⁺ T-cells at several DC to T-cell ratios. The T-cell stimulation assay is carried out for about 7 days to identify any T-cell proliferation. At the end of the 7-day period, the cells are analyzed by flow cytometry, identifying both cell surface markers and CFSE dilution as an indicator of proliferation. Control samples for the T-cell proliferation assay include samples of T-cells only and T-cells mixed with DCs collected from a MES model that was not primed with antigen.

### Example 26

Mature FDCs are immobile and reside in the light zones of GCs where they play an important role in attracting B cells and establishing the GC architecture (FIG. 36). In embodiments of the present invention, to construct a 3D in vitro MTE that includes GCs, FDCs are seeded into an ECM (e.g., one comprising collagen) to attract B and T cells and form FDC-B cell-T cell clusters, as they do in vivo. FDCs cultured on plastic fail to adhere, remain rounded, and are unable to form networks. In contrast, FDCs placed on collagen-coated plates, attached to the matrix, regenerated processes, and generated networks with features in common with the networks seen in vivo. The ECM can comprise collagen and other ECM proteins, such as biglycan, laminin, or fibronectin. The ability of FDCs to bind these collagen, collagen-associated molecules, and mixtures thereof, may explain why these cells are fixed to the lymph node matrix and do not circulate as other immune system cells do. CXCL 13, a chemokine secreted by FDCs, has been shown to attract human B cells and T cells into follicular zones. Additionally, GC B cells are activated and express a unique phenotype, PNA⁺, GL-7⁺, CD95^{hi} and CD23^{lo} and segregate into light zones where they are centrocytes and into dark zones where they are centroblasts.

### Example 27

A schematic representation of the MTE model, an embodiment of the present invention, is shown in FIGS. 39 and 40. In other embodiments, exogenous chemokines such as BCA-1 (CXCL 13) and CCL21, can be used to stimulate lymphocyte migration (Kanemitsu et al. (2005) Blood 106, 2613-2618; Vermi et al. (2005) Blood 107, 453-462).

The above description is for the purpose of teaching the person of ordinary skill in the art how to practice the present invention, and it is not intended to detail all those obvious modifications and variations of it that will become apparent to the skilled worker upon reading the description. It is intended, however, that all such obvious modifications and variations be included within the scope of the present invention, which is defined by the following claims. The claims are intended to cover the claimed components and steps in any sequence that is effective to meet the objectives there intended, unless the context specifically indicates the contrary.

1. A mucosal tissue equivalent system comprising:
fibroblasts embedded in a matrix;
an endothelial layer attached to one side of said matrix; and
an epithelial layer attached to the other side of said matrix.

2. A mucosal tissue equivalent system comprising:
fibroblasts embedded in a matrix;
an endothelium attached to one side of said matrix; and
an epithelium attached to the other side of said matrix.

3. A mucosal tissue equivalent system comprising:
fibroblasts embedded in a matrix;
a vascular endothelium attached to one side of said matrix; and
a mucosal epithelium attached to the other side of said matrix.

4. A mucosal tissue equivalent system comprising:
fibroblasts embedded in a matrix;
a confluent vascular endothelium attached to one side of said matrix; and
a mucosal epithelium attached to the other side of said matrix.

5. An artificial immune system comprising:
a mucosal tissue equivalent system; and
a three-dimensional artificial lymphoid tissue, comprising a matrix and a plurality of lymphocytes and leukocytes.

6. The mucosal tissue equivalent system of claim 1, wherein said fibroblast-embedded matrix further comprises cells selected from the group consisting of T cells, B cells, macrophages, monocytes, mast cells, dendritic cells, and follicular dendritic cells.

7. The mucosal tissue equivalent system of claim 1, wherein said system further comprises a lymphoid follicle.

8. The mucosal tissue equivalent system of claim 1, wherein said system further comprises a germinal center.

9. The mucosal tissue equivalent system of claim 1, wherein said system is organized in a well.

10. The mucosal tissue equivalent system of claim 1, wherein said system is organized in a multi-well format.

11. The mucosal tissue equivalent system of claim 1, wherein said epithelial layer is selected from the group consisting of nasal epithelium, oral epithelium, respiratory epithelium, gastrointestinal epithelium, conjunctival epithelium, and urogenital epithelium.

12. The mucosal tissue equivalent system of claim 1, wherein said matrix is selected from the group consisting of a collagen membrane, hydrogels, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM (such as small intestine submucosa and urinary bladder mucosa).

13. The mucosal tissue equivalent system of claim 1, wherein said decellularized ECM is selected from the group consisting of small intestine submucosa and urinary bladder mucosa.

14. A method of developing an in vitro lymphoid follicle in a synthetic or natural extracellular matrix (ECM) material, comprising placing follicular dendritic cells in the synthetic or natural extracellular matrix material in conditions in which they can develop a three-dimensional germinal center.

15. A method of developing an in vitro germinal center in a synthetic or natural extracellular matrix (ECM) material, comprising placing follicular dendritic cells in the synthetic or natural extracellular matrix material in conditions in which they can develop a three-dimensional germinal center.

16. A method of developing an in vitro lymphoid follicle in a synthetic or natural extracellular matrix (ECM) material, comprising placing follicular dendritic cells in the synthetic or natural extracellular matrix material in conditions in which they can develop a three-dimensional lymphoid follicle.

17. The method of claim 14, wherein the synthetic or natural extracellular matrix is selected from the group consisting of a collagen cushion, microcarriers, inverted colloid crystal matrices, collagen membranes, hydrogels, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM.

18. A method for using the mucosal tissue equivalent system of claim 1 for testing the immunogenicity of an agent, said method comprising:
applying the antigen to the epithelial cells in the epithelial layer; and
analyzing the immune response.

19. A method for using the mucosal tissue equivalent system of claim 3 for testing the immunogenicity of an agent, said method comprising:
applying the antigen to the mucosal epithelial cells in the mucosal epithelium; and
analyzing the immune response.

20. The method of claim 19, wherein said agent is selected from the group consisting of vaccines, respiratory pathogens, allergens, drugs, and immunogens.

21. A method of determining whether a patient is a poor or non-responder to a vaccine, comprising:
preparing a mucosal tissue equivalent system comprising:
embedding fibroblasts from said patient in a matrix;
attaching endothelial cells from said patient to one side of said matrix; and
attaching epithelial cells from said patient to the other side of said matrix; and
administering the vaccine to said epithelial cells, and analyzing the immune response to said vaccine.

22. A method of determining whether a patient is a poor or non-responder to a vaccine, comprising:
preparing a mucosal tissue equivalent system comprising:
embedding fibroblasts from said patient in a matrix;
attaching vascular endothelium cells from said patient to one side of said matrix; and
attaching mucosal epithelium cells from said patient to the other side of said matrix; and
administering the vaccine to said mucosal epithelium cells, and analyzing the immune response to said vaccine.

23. The method of claim 21, wherein said fibroblast-embedded matrix further comprises cells selected from the group consisting of T cells, B cells, macrophages, monocytes, mast cells, dendritic cells, and follicular dendritic cells.

24. The method of claim 21, wherein said system further comprises a lymphoid follicle center.

25. The method of claim 21, wherein said system further comprises a germinal center.

26. The method of claim 21, wherein said system is organized in a well.

27. The method of claim 21, wherein said system is organized in a multi-well format.

28. The method of claim 22, wherein said mucosal epithelium is selected from the group consisting of nasal epithelium, oral epithelium, respiratory epithelium, gastrointestinal epithelium, conjuctival epithelium and urogenital epithelium.

29. The method of claim 21, wherein said matrix is selected from the group consisting of a collagen membrane, hydrogel, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM.

30. A method of identifying agents that can convert a patient that is a poor or non-responder to a vaccine to a good responder to said vaccine, comprising:
preparing a mucosal tissue equivalent system comprising:
embedding fibroblasts from said patient in a matrix;
attaching endothelial cells from said patient to one side of said matrix; and
attaching epithelial cells from said patient to the other side of said matrix;
administering an immunomodulator to said epithelial cells;
administering said vaccine to said epithelial cells; and
analyzing said patient's response to said vaccine to determine whether said patient has been converted to a good responder to said vaccine.

31. A method of identifying agents that can convert a patient that is a poor or non-responder to a vaccine to a good responder to said vaccine, comprising:
preparing a mucosal tissue equivalent system comprising:
embedding fibroblasts from said patient in a matrix;
attaching vascular endothelium cells from said patient to one side of said matrix; and
attaching mucosal epithelium cells from said patient to the other side of said matrix;
administering an immunomodulator to said mucosal epithelium cells;
administering said vaccine to said mucosal epithelium cells; and
analyzing said patient's response to said vaccine to determine whether said patient has been converted to a good responder to said vaccine.

32. The method of claim 30, wherein said fibroblast embedded matrix further comprises cells selected from the group consisting of T cells, B cells, macrophages, monocytes, mast cells, dendritic cells and follicular dendritic cells.

33. The method of claim 30, wherein said system further comprises a lymphoid follicle.

34. The method of claim 30, wherein said system further comprises a germinal center.

35. The method of claim 30, wherein said system is organized in a well.

36. The method of claim 30, wherein said system is organized in a multi-well format.

37. The method of claim 31, wherein said mucosal epithelium is selected from the group consisting of nasal epithelium, oral epithelium, respiratory epithelium, gastrointestinal epithelium, conjuctival epithelium and urogenital epithelium.

38. The method of claim 30, wherein said matrix is selected from the group consisting of a collagen membrane, hydrogel, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM.

39. A method for identifying agents useful for treating an antibody-mediated autoimmune disorder in a patient comprising:
preparing a mucosal tissue equivalent system comprising:
embedding fibroblasts from said patient in a matrix;
attaching vascular endothelium cells from said patient to one side of said matrix; and
attaching mucosal epithelium cells from said patient to the other side of said matrix;
administering an agent to said mucosal epithelium cells; and
quantifying the amount of autoimmune antibodies present in the mucosal tissue equivalent system.

40. A method for identifying agents useful for treating an antibody-mediated autoimmune disorder in a patient comprising:
preparing a mucosal tissue equivalent system comprising:
embedding fibroblasts from said patient in a matrix;
attaching endothelial cells from said patient to one side of said matrix; and
attaching epithelial cells from said patient to the other side of said matrix;
administering an agent to said epithelial cells; and
quantifying the amount of autoimmune antibodies present in the mucosal tissue equivalent system.

41. The method of claim 40, wherein said fibroblast embedded matrix further comprises cells selected from the group consisting of T cells, B cells, macrophages, monocytes, mast cells, dendritic cells, and follicular dendritic cells.

42. The method of claim 40, wherein said system further comprises a lymphoid follicle.

43. The method of claim 40, wherein said system further comprises a germinal center.

44. The method of claim 40, wherein said system is organized in a well.

45. The method of claim 40, wherein said system is organized in a multi-well format.

46. The method of claim 39, wherein said mucosal epithelium is selected from the group consisting of nasal epithelium, oral epithelium, respiratory epithelium, gastrointestinal epithelium, conjuctival epithelium and urogenital epithelium.

47. The method of claim 40, wherein said matrix is selected from the group consisting of a collagen membrane, hydrogel, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM.

48. A method of preparing a mucosal tissue equivalent system comprising:
seeding fibroblasts on a matrix;
seeding mucosal epithelial cells on one side of said matrix; and
seeding vascular endothelial cells on the other side of said matrix.

49. A method of preparing a mucosal tissue equivalent system comprising:
seeding fibroblasts on a matrix;
seeding epithelial cells on one side of said matrix; and
seeding endothelial cells on the other side of said matrix.

50. The method of claim 49, wherein said fibroblast-embedded matrix further comprises cells selected from the group consisting of T cells, B cells, macrophages, monocytes, mast cells, dendritic cells, and follicular dendritic cells.

51. The method of claim 49, wherein said system further comprises a lymphoid follicle.

52. The method of claim 49, wherein said system further comprises a germinal center.

53. The method of claim 49, wherein said system is organized in a well.

54. The method of claim 49, wherein said system is organized in a multi-well format.

55. The method of claim 48, wherein said mucosal epithelium is selected from the group consisting of nasal epithelium, oral epithelium, respiratory epithelium, gastrointestinal epithelium, conjuctival epithelium and urogenital epithelium.

56. The method of claim 49, wherein said matrix is selected from the group consisting of a collagen membrane, hydrogel, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM.

57. The mucosal tissue equivalent system of claim 1, wherein said system is organized in a flow-based bioreactor.

58. The mucosal tissue equivalent system of claim 1, wherein said system is organized in a microfluidic flow-based bioreactor.

59. The method of claim 15, wherein the synthetic or natural extracellular matrix is selected from the group consisting of a collagen cushion, microcarriers, inverted colloid crystal matrices, collagen membranes, hydrogels, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM.

60. The method of claim 16, wherein the synthetic or natural extracellular matrix is selected from the group consisting of a collagen cushion, microcarriers, inverted colloid crystal matrices, collagen membranes, hydrogels, poly(methyl methacrylate, poly(lactide-co-glycolide), polytetrafluoroethylene, poly(ethylene glycol dimethacrylate) hydrogels (PEGDA or PEGDMA), poly(ethylene oxide), poly(propylene fumarate-co-ethylene glycol) (PPF-PEG), hyaluronic acid hydrogels, calf skin gelatin, fibrinogen, thrombin, and decellularized ECM.
